(19) United States
(12) Patent Application Publication
(10) Pub. No.: US 2024/0148901 A1
(43) Pub. Date: May 9, 2024

(54) COMPOSITIONS AND METHODS FOR PROPHYLAXIS OF HIV

(71) Applicant: GEORGIA TECH RESEARCH CORPORATION, Atlanta, GA (US)

(72) Inventors: Philip J. SANTAGELO, Smyrna, GA (US); Kevin LINDSAY, Atlanta, GA (US); Daryll VANOVER, Duluth, GA (US)

(73) Assignee: GEORGIA TECH RESEARCH CORPORATION, Atlanta, GA (US)

(21) Appl. No.: 17/772,985

(22) PCT Filed: Oct. 28, 2020
(86) PCT No.: PCT/US2020/057720

§ 371 Date: Apr. 28, 2022

Related U.S. Application Data
(60) Provisional application No. 62/926,779, filed on Oct. 28, 2019.

(51) Int. Cl. A61K 48/00; A61K 9/12; A61P 31/18; C07K 16/10; C12N 15/85

(52) U.S. Cl. CPC A61K 48/005; A61K 9/12; A61P 31/18; C07K 16/1063; C12N 15/85

(57) ABSTRACT

Prophylactic compositions for inhibiting or reducing HIV infection and methods of their use are provided. One embodiment provides an antibody or an antigen binding fragment thereof that specifically binds to an HIV protein, for example gp120, and Cinhibits or reduces the ability of the HIV vims to infect human cells. One embodiment provides a PGT121 antibody or antigen binding fragment thereof modified to contain a GPI membrane anchor, wherein the antibody specifically binds to an HIV protein. In some embodiments the GPI membrane anchor is in the heavy chain of the antibody or antigen fragment thereof. In some aspects the antibody or antigen binding fragment specifically to gp120 or gp41.

### CROSS-REFERENCE TO RELATED APPLICATIONS

This application claims benefit of and priority to U.S. Provisional Patent Application No. 62/926,779 filed on Oct. 28, 2019, which is incorporated by reference in its entirety.

### TECHNICAL FIELD OF THE INVENTION

Aspects of the invention are generally directed to compositions and methods for the prophylaxis of human immune deficiency virus.

### BACKGROUND OF THE INVENTION

Human immunodeficiency virus (HIV) remains a substantial public health burden worldwide, with 36 million people infected and 1.8 million new cases per year 1. >90% of infections occur via sexual contact and the estimated probability of infection of the female genital tract (FRT) is 1 in 200-2000 per coital act, depending on the viral burden in the donor (Brandenberg, O. F. et al. Predicting HIV-1 transmission and antibody neutralization efficacy in vivo from stoichiometric parameters. PLOS Pathog. 13, e1006313 (2017); Miller, W. C., Rosenberg, N. E., Rutstein, S. E. & Powers, K. A. Role of acute and early Hiv infection in the sexual transmission of Hiv. Curr. Opin. Hiv Aids 5, 277-282 (2010)). Recipient factors providing protection include mucus, antimicrobials present in genital secretions, intact tight junctions between epithelial cells, lactobacillus dominated microbiome, and genetic factors4. The increased use of anti-retroviral therapy (ART) has markedly improved the outlook of HIV infected patients and the recently approved pre-exposure (PrEP), and post-exposure prophylaxis regimens are >90% efficacious. PrEP however, consists of a daily use regimen, with a high probability of non- or partial adherence, the potential for side effects, expensive cost, and a lack of coverage against other sexually transmitted infections (STIs). Therefore, alternative approaches suitable for self-application in the treatment and prevention of HIV could prove useful.

Virus introduced into the FRT lumen via ejaculate or released from infected donor cells rapidly permeates the vaginocervical epithelium by passive diffusion, perhaps as quickly as within 30 minutes (Stieh, D. J. et al. Vaginal Challenge with an SIV-Based Dual Reporter System Reveals That Infection Can Occur throughout the Upper and Lower Female Reproductive Tract. PLOS Pathog. 10, e1004440 (2014); Anderson, D. J. Finally, a macaque model for cell-associated SIV/HIV vaginal transmission. J. Infect. Dis. 202, 333-336 (2010)) and reaches systemic lymph nodes in less than 24 hours (Barouch, D. H. et al. Rapid Inflammasome Activation following Mucosal SIV Infection of Rhesus Monkeys. Cell 165, 656-667 (2016)). Thus, intervention strategies should counteract initial virus seeding and replication in the lower FRT and prevent trafficking of virions to regional lymph nodes.

HIV envelope (Env) is expressed on the surface of virions and infected cells as a trimeric glycoprotein of two non-covalently associated subunits, gp120 and gp41, responsible for binding CD4 receptors on target cells and mediating membrane fusion, respectively. For genital IgG, it appears that neutralization of Env is a major factor in protection from viral acquisition8.

Broadly neutralizing antibodies (bNAbs), characterized by their ability to neutralize a broad assortment of HIV strains with high potencies, have been isolated from a small subset of infected individuals (Freund, N. T. et al. Coexistence of potent HIV-1 broadly neutralizing antibodies and antibody-sensitive viruses in a viremic controller. Sci. Transl. Med. 9, eaa12144 (2017)). Passive immunoprophylaxis by parenteral administration of bNAbs has been shown to prevent infection (Julg, B. et al. Protective Efficacy of Broadly Neutralizing Antibodies with Incomplete Neutralization Activity against Simian-Human Immunodeficiency Virus in Rhesus Monkeys. J. Virol. 91, e01187-17 (2017); Julg, B. et al. Protection against a mixed SHIV challenge by a broadly neutralizing antibody cocktail. Sci. Transl. Med. 9, eaao4235 (2017); Hessell, A. J. et al. Broadly Neutralizing Human Anti-HIV Antibody 2G12 Is Effective in Protection against Mucosal SHIV Challenge Even at Low Serum Neutralizing Titers. PLOS Pathog. 5, e1000433 (2009)) even post-exposure (Hessell, A. J. et al. Early short-term treatment with neutralizing human monoclonal antibodies halts SHIV infection in infant macaques. Nat. Med. 22, 362-368 (2016)) and is currently being tested for its ability to reduce viral reservoirs in HIV infected patients (Caskey, M. et al. Viraemia suppressed in HIV-1-infected humans by broadly neutralizing antibody 3BNC117. Nature 522, 487-491 (2015); Caskey, M. et al. Antibody 10-1074 suppresses viremia in HIV-1-infected individuals. Nat. Med. 23, 185-191 (2017)) and suppress viral recrudescence after ART interruption (Scheid, J. F. et al. HIV-1 antibody 3BNC117 suppresses viral rebound in humans during treatment interruption. Nature 535, 556-560 (2016)). In one study, administration of bNAbs to monkeys during acute SHIV infection led to long-lasting CD8+ T cell immunity that suppressed virus long after administered antibody titers were undetectable (Nishimura, Y. et al. Early antibody therapy can induce long-lasting immunity to SHIV. Nature 543, 559-563 (2017)).

To protect patients from mucosal HIV infection, antibodies delivered parenterally must reach the genital compartment through transudation from the serum. It has been estimated that the concentration of antibody in the serum is approximately 90-fold higher than in vaginal secretions (Brandenberg, 0. F. et al. Predicting HIV-1 transmission and antibody neutralization efficacy in vivo from stoichiometric parameters. PLOS Pathog. 13, e1006313 (2017)). The amount of bNAb required in genital secretions to prevent infection decreases with higher binding affinities. For the bNAb PGT121, with a relatively high binding affinity (k_{D} =0.086 nM), genital secretion concentrations as low as 30 ng/mL are estimated to provide neutralizing protection. There also appears to be a time delay between the peak serum concentration of injected antibody (which is almost immediately) and the peak concentration in the vaginal secretions, although the magnitude of this delay is dependent on the specific antibody 12. Hessell, A. J. et al. Broadly Neutralizing Human Anti-HIV Antibody 2G12 Is Effective in Protection against Mucosal SHIV Challenge Even at Low Serum Neutralizing Titers. PLOS Pathog. 5, e1000433 (2009)). While systemically administered bNAbs have demonstrated promising anti-viral properties, the large doses and uncertain time required for genital secretions and tissues to reach sufficient concentrations suggest that local application of bNAbs could provide a viable alternative. Direct vaginal application of bNAbs before challenge protected NHPs19, but the half-life of such an approach is likely on the order of several hours (Sherwood, J. K. Residence half-life of IgG administered topically to the mouse vagina. Biol. Reprod. 54, 264-269 (1996)).

Therefore, it is an object of the invention to provide compositions and methods for prophylactic treatment to inhibit or reduce HIV invention.

### SUMMARY OF THE INVENTION

Prophylactic compositions for inhibiting or reducing HIV infection and methods of their use are provided. One embodiment provides an antibody or an antigen binding fragment thereof that specifically binds to an HIV protein, for example gp120, and inhibits or reduces the ability of the HIV virus to infect human cells. One embodiment provides a PGT121 antibody or antigen binding fragment thereof modified to contain a GPI membrane anchor, wherein the antibody specifically binds to an HIV protein. In some embodiments the GPI membrane anchor is in the heavy chain of the antibody or antigen fragment thereof In some aspects the antibody or antigen binding fragment specifically to gp120 or gp41.

One embodiment provides a vector comprising SEQ ID Nos 9-13. In one embodiment the vector is an mRNA.

One embodiment provides a vector containing SEQ ID Nos:14-22. In one embodiment the vector is mRNA.

One embodiment provides a vector containing SEQ ID Nos:23-24. In one embodiment the vector is mRNA.

One embodiment provides an antibody or an antigen binding fragment thereof comprising a heavy chain amino acid sequence according to SEQ ID NO:27, a GPI membrane anchor have the amino acid sequence according to SEQ ID NO:28, and a light chain amino acid sequence according to SEQ ID NO:30.

One embodiment provides an antibody or antigen binding fragment thereof having a first heavy chain with an amino acid sequence according to SEQ ID NO:32, a second heavy chain having an amino acid sequence according to SEQ ID NO:34, a GPI membrane anchor with an animo acid sequence according to 35, a first light chain with an amino acid sequence according to 37, and a second light chain having an amino acid sequence according to SEQ ID NO:39. One embodiment provides an antibody or an antigen binding fragment thereof having a heavy chain with an amino acid sequence according to SEQ ID NO:41 and a GPI membrane anchor according to SEQ ID NO:42.

Another embodiment provides a recombinant genetic construct that encodes an antibody or an antigen binding fragment thereof that specifically binds to an HIV protein, for example gp120, and inhibits or reduces the ability of the HIV virus to infect human cells wherein the antibody is modified to include a GPI membrane anchor. In one embodiment, the antibody is a PGT121 antibody as shown in FIG. 14, wherein the antibody is modified to include a GPI anchor, for example in the heavy chain of the antibody. In some embodiments, the genetic construct is an RNA construct, for example a mRNA construct. In some embodiments, the mRNA construct is fully modified with complete substitution of uridine with n1-methyl-pseudouridine.

Another embodiment provides a pharmaceutical composition consisting of the mRNA construct that encodes an antibody or an antigen binding fragment thereof that specifically binds to an HIV protein, for example gp120, and inhibits or reduces the ability of the HIV virus to infect human cells wherein the antibody is modified to include a GPI membrane anchor and water. In some embodiments, the pharmaceutical composition also contains a buffer.

One embodiment provides a method for prophylactically inhibiting or reducing HIV infection of a female subject comprising the steps of transfecting cervicovaginal epithelial cells in the subject with the construct encoding a PGT121 antibody or antigen binding fragment thereof wherein the antibody or antigen binding fragment is modified to include a GPI membrane anchor. In some embodiments, the construct is an mRNA construct. It will be appreciated that any of the vectors disclosed herein can be used in this method.

Another embodiment provides a method for prophylactically inhibiting or reducing HIV infection in a female subject comprising the step of administering the pharmaceutical composition consisting of the mRNA construct encoding a that encodes an antibody or an antigen binding fragment thereof that specifically binds to an HIV protein, for example gp120, and inhibits or reduces the ability of the HIV virus to infect human cells wherein the antibody is modified to include a GPI membrane anchor and water. It will be appreciated that any of the vectors disclosed herein can be used in this method.

One embodiment provides an antibody comprising a heavy chain encoded by a nucleic acid having 85%, 90%, 95%, 99%, or 100% sequence identity to SEQ ID NO:1 and a light chain encoded by nucleic acid having 85%, 90%, 95%, 99%, or 100% sequence identity to SEQ ID NO:2.

Another embodiment provides a kit comprising a container comprising the that encodes an antibody or an antigen binding fragment thereof that specifically binds to an HIV protein, for example gp120, and inhibits or reduces the ability of the HIV virus to infect human cells wherein the antibody is modified to include a GPI membrane anchor and an aerosol delivery device. In one embodiment, the antibody is a PGT121 antibody as shown in FIG. 14, wherein the antibody is modified to include a GPI anchor, for example in the heavy chain of the antibody. In some embodiments, the genetic construct is an RNA construct, for example a mRNA construct. In some embodiments, the mRNA construct is fully modified with complete substitution of uridine with n1-methyl-pseudouridine. In some embodiments the delivery device is an atomizer or a dual-chamber syringe containing lyophilized mRNA and water and an atomizer suitable for self-insertion into the FRT.

### BRIEF DESCRIPTION OF THE DRAWINGS

FIGS. 1A1-1A3 are fluorescent micrographs of Vero cells that were transfected via aerosol with H₂O (control), GFP-encoding mRNA, or GFP-encoding plasmid complexed with lipofectamine 2000 (pDNA+L2k). Cells were fixed and imaged for GFP (green) at 24 h. Scale bars represent 30 pm. FIG. 1B is a graph showing the quantification of the GFP MFI on a per-cell basis, for each transfection condition. FIGS. 1C1-1C10 are fluorescent micrographs showing that dye-labeled mRNA was delivered via aerosol to Vero cells and fixed at 30 s, 5 m, 1 h, 2 h, and 6 h post-transfection. Cells were then stained with a pan-endosomal marker cocktail including: CD63, clathrin, caveolin, EEA1, and LAMP-1. Scale bars represent 10 pm. FIG. 1D is a graph showing the extent of spatial overlap between the endosomal markers and mRNA at each time point, up to 1 hour. The Mender's overlap coefficient reflects the proportion of mRNA that overlaps with the endosomal markers. FIG. 1E is a graph showing the proportion of mRNA overlapping with the endosomal markers from 1 hour to 6 hours, contrast enhanced on a different scale than the acute time points in part (1D). FIG. 1F shows that in live sheep, 250 pg of firefly luciferase mRNA in H₂O was delivered to the cervix as a single dose with either a high-pressure syringe or via aerosolizer. A speculum was used to visualize the cervix in sedated sheep during transfection. Twenty-four hours post-transfection, the animals were euthanized and subjected to necropsy. Luciferin was added to isolated cervix and luminescence quantified as the average radiance (p/s/cm²/sr) per animal via an IVIS Lumina imaging system. Statistical comparison was performed using two tailed Mann-Whitney non-parametric analyses. For all panels, p<0.05, ****, p<0.00005.

FIG. 2A is a schematic of the PGT121-NanoLucg fusion protein, in secreted and anchored forms. NanoLuc® was fused to the 3′end of the kappa light chains (2 per antibody molecule). In the case of the membrane-anchored PGT121, a GPI anchor was fused to the 3′ end of the heavy chain. Total MW of anchored PGT121-NanoLuc® was 195 kDa. FIG. 2B are microfluorographs of HEK293 cells that were transfected with 1 pg of PGT121 mRNA, delivered at a 4:1 ratio of heavy chain to light chain with lipofectamine 2000 (top row). Respiratory syncytial virus neutralizing anchored Palivizumab (aPali) antibody mRNA was used as a control comparison (bottom row). Twenty-four hours later, DyLight 680-labeled SHIV particles (red) were incubated with the transfected cells for four hours without agitation. After washing 3×, the cells were fixed and immunostained with an anti-human secondary antibody (green). FIG. 2C is a graph showing the average percentage overlap between SHIV virus and anchored antibodies from panel (2B), using 30 cells per condition. The error bars represent the mean±95% Cl. FIGS. 2D1 and 2D2 are graphs showing anchored and secreted PGT121 was produced in Vero cells and purified. The mRNA-expressed antibodies were compared against parental PGT121 for SHIV neutralization with either a Glade B or Glade C SHIV isolate. Error bars represent SD. FIGS. 2E1 and 2E2 are graphs showing anchored and secreted PGT121 fused to NanoLuc® was produced in Vero cells and purified. The mRNA-expressed NanoLuc® fusion antibodies were compared against parental PGT121 for SHIV neutralization with either a Glade B or Glade C SHIV isolate. Error bars represent SD. FIGS. 2F1 and 2F2 are graphs showing mRNA-expressed anchored or secreted PGT121 either with or without NanoLuc® was purified, diluted, and tested for ADCC against SHIV162p3 (Clade B). Complete dilution series (left) and percent maximal ADCC (right) are shown. Error bars represent SD. Percent maximum ADCC compared by Kruskal-Wallis. FIGS. 2G1 and 2G2 are graphs showing the same antibodies from part F were purified, diluted, and tested for ADCC against SHIV2871Nip (Clade C). Complete dilution series (left) and percent maximal ADCC (right) are shown. Error bars represent SD. Percent maximum ADCC compared by Kruskal-Wallis.

FIG. 3A is a panel of fluorescent images of cervicovaginal epithelium from sheep 410, 426, 463, 404, 406, and 407 showing a single 250 pg or 750 pg dose of aPGT121-NanoLuc® that was delivered by aerosol to the cervix. After 24 hours, sheep were euthanized, FRT excised, luciferin added, and luminescence measured via IVIS. Representative images demonstrating the intensity in each animal are displayed. FIG. 3B is a control image of cervicovaginal epithelium from sheep 415 processed in the same manner as in 3A. FIG. 3C is a graph showing the average radiance at the cervix over the average radiance in the control animal, for each mRNA dosing group. Error bars represent the ±95% Cl. In FIGS. 3D1-3D3 aerosolized dye-labeled mRNA was delivered to 2 animals as previously described. After 24 hours, the cervix was excised and processed for immunofluorescence tissue imaging. DAPI—cell nuclei, White—aPGT121 mRNA, Green—anti-NanoLuc® antibody. Section scale bars represent 10 pm. FIG. 3E is a panel of images showing data after aPGT121-NanoLuc® mRNA was sprayed in four consecutive 750 pg doses, beginning at the cervix (marked as ‘1’) and retracting caudally to distal vagina (‘4’). Bottom images are immunofluorescence images.

FIGS. 4A-4B are graphs aPGT121 persists in vaginal secretions out to 28 days post-transfection. Three sheep were transfected with two doses of 750 fag of mRNA encoding for aPGT121, while another two sheep were transfected with an equal mass of sPGT121 mRNA. Vaginal secretions were collected at 1, 7, 14, 21, and 28 days post-transfection. FIG. 4A shows longitudinal sampling of PGT121 concentrations over time, for all animals. The horizontal asymptote represents the limit of detection. FIG. 4C shows antibody concentrations in vaginal secretions that were quantified via Western Blot analysis, using a standard curve of purified PGT121-NanoLuc® protein. The expected size of aPGT121-NanoLuc® was confirmed to be around 195 kDa. For secreted antibody, the LC uncoupled from the HC, resulting in a 54 kDa band.

FIGS. 5A-5E show that aPGT121 persists in the FRT mucosa at 28 days post-transfection. FIG. 5A shows IVIS imaging of the excised lower FRT at 14 days and 28 days post-transfection. 28-day samples for sPGT121 transfected animals were not collected due to the low signal observed at 14 days. FIG. 5B is a graph showing average radiance of the secreted and anchored PGT121-NanoLucg constructs. Mann-Whitney non-parametric analysis was used to compare the two groups. FIG. 4C is an image showing that after 28 days, tissue samples were excised under the guidance of IVIS signal, snap-frozen, and pulverized for downstream western blot analysis. FIG. 4D is a graph showing day 28 post-transfection aPGT121 concentrations in excised cervix, vagina, uterus, and caudal vagina were estimated using quantitative western blot. Five animals, euthanized at 28 days, were used in total. FIG. 4E is a Western blot demonstrating the characteristic aPGT121 band at 195 kDa for all regions of the FRT of one treated sheep, compared to control cervix.

FIGS. 6A1-6D show monitoring of ⁶⁴Cu radiolabeled aPGT121 mRNA after aerosolized FRT delivery. In FIGS. 6A1-6C two 125 fag doses of ⁶⁴Cu radiolabeled aPGT121 mRNA were delivered via aerosol to first the cervix, then −3-4 cm caudally in the vagina. A total of 200 pCi of 64Cu was administered. PET/CT imaging over three days was used to monitor mRNA biodistribution. FIG. 6A1-6A12 are representative PET/CT images of the abdomen and pelvis from 70 minutes to 72 hours. Contrast enhancement was adjusted to reflect the high SUV signals within the FRT. FIG. 6B is graph showing the total SUV in the FRT over time. FIG. 6C is a graph showing the ratio of the total SUV in the FRT to the total SUV contained within the entire body. FIG. 6D1-6D8 are whole-body PET/CT images of macaque RVg13 at 70 minutes and 24 hours post-transfection. Numbers in white represent the total SUV within the nearby organ. Contrast enhancement was adjusted to allow visualization of draining lymph nodes.

FIGS. 7A-7B show aPGT121 mRNA protects rhesus macaque biopsy explants from SHIV162p3 challenge and is neutralizing in genital secretions. FIG. 7A is a graph showing 250 (n=2), 400 (n=1), or 1000 pg (n=1) of aerosolized aPGT121 mRNA was delivered to the ectocervix and rostra! vagina of rhesus macaques. From the 250 pg group, biopsy explants were collected from the endocervix and vagina at 24 hours post-transfection, while from the 400 and 1000 pg groups, two biopsies were collected from each of the indicated regions at 24 hours post transfection. Dotted vertical line separates the two animals in the 250 pg group. Biopsies were then challenged with SHIV162p3 virus. In FIG. 7B the neutralization activity of genital secretions at 4 h, 24 h, 48 h, 72 h, and 1-week post-transfection against Clade B and C SHIV strains was evaluated using the in vitro TZM-bl assay. All macaques were treated with 250 pg of aPGT121 mRNA. Macaque RCol3 was only transfected with aPGT121 heavy chain (i.e., no light chain). The dashed horizontal line represents the lower limit of detection. Complete dilution series neutralization data can be found in FIG. 12.

FIG. 8A is a photograph of a representative in vitro aerosol apparatus. Height was adjusted using a scissor-lift lab jack. Teleflex was held using clamps and actuated by hand. FIG. 8B is a photograph of an in vivo Teleflex apparatus with speculum. Teleflex syringe was modified using flat and conical rubber washer to allow for consistent centering and distance when placed in the speculum. The speculum was a cut and sanded piece of plastic tubing. FIG. 8C is a photograph showing treatment of sheep in field. Sheep were sedated and placed in dorsal recumbency to allow for easy mRNA application.

FIG. 9 is graph showing an HIV gp120 ELISA using PGT121:NanoLuc® Luminescence. Luminescence of a range of concentrations of purified PGT121:NanoLuc® were either measured in solution (PBS) or after capture via gp120 coated plate (ELISA). Linear regression was performed on log-log transformed data.

FIGS. 10A1-10A4 and 10B1-10B4 fluorescence micrographs showing PGT121 LC and HC localization to cell surface after mRNA transfection. FIGS. 10A1-10A4 show HEK293 cells that were transfected using lug of total synthetic PGT121 mRNA, in the following conditions: 1) as a 4:1 ratio of HC to LC encoding transcripts; 2) HC encoding transcripts only; or 3) LC encoding transcripts only. 24 hours post-transfection. The cells were fixed, not permeabilized, and immunostained with anti-human antibody (Jackson). White—anti-human antibody (Jackson), Blue—DAPI. Scale bars are 10 μm. FIGS. 10B1-10B4 show HEK293 cells that were transfected with lug of HC and LC PGT121 mRNA, at a 4:1 ratio. After 24 hours, the cells were fixed and immunostained with anti-human Fc and anti-kappa light chain antibodies. Colocalization coefficients indicate that 98% of the LC protein overlaps with HC protein. Green (not shown)—anti-human antibody (Jackson), Red (not shown)—anti-kappa light chain (BD), Blue (not shown)—DAPI. Scale bars are 10 μm.

FIGS. 11A-11D show a Quantitative Western methodology for aPGT121-NanoLucg. Schematic of quantitative western blot protocol. On each gel, a standard of purified PGT121 was loaded and quantified by densitometry. A linear regression was then run on these values, and the densitometry of the samples was interpolated to determine the amount of PGT121 in the loaded sample. Samples were loaded onto the same gel in all cases. FIG. 11A is a Western blot of a standard of purified PGT121. FIG. 11B is a graph showing linear regression on band signal densitometry. FIG. 11C is a Western blot of samples. FIG. 11D is a graph of PG7121 concentration (μg/mL) versus days post transfection sheep numbers 420, 456, and 461.

FIG. 12 is a graph of baseline vaginal explant challenges. Cervical and vaginal biopsies taken prior to mRNA delivery were challenged with SHIV162p3. Points indicate TZM assays on days 0, 4, 7, and 10 for each biopsy.

FIGS. 13A-13F show a vaginal secretion neutralization dilution series. The neutralization activity of genital secretions from animals treated with 250 μg of aPGT121 mRNA at 4 h, 24 h, 48 h, 72 h, and 1-week post-transfection against Clade B and C SHIV strains was evaluated using the in vitro TZM-bl assay. Macaque RCo13 was only transfected with aPGT121 heavy chain (i.e., no light chain).

FIG. 14 shows the amino acid sequences of PGT121, 122, and 123 light chain and heavy chains taken from Julien J P, Sok D, Khayat R, Lee J H, Doores K J, et al. (2013) Broadly Neutralizing Antibody PGT121 Allosterically Modulates CD4 Binding via Recognition of the HIV-1 gp120 V3 Base and Multiple Surrounding Glycans. PLoS Pathog 9(5): e1003342. doi:10.1371/journal.ppat.1003342 which is incorporated by reference in its entirety.

### DETAILED DESCRIPTION OF THE INVENTION

### I. Definitions

A vector or recombinant genetic construct that can be used herein includes, but is not limited to, a viral vector, a plasmid, a RNA vector or a linear or circular DNA or RNA molecule which may include a chromosomal, nonchromosomal, semi-synthetic or synthetic DNA. Some vectors are those capable of autonomous replication (episomal vector) and/or expression of nucleic acids to which they are linked (expression vectors). Large numbers of suitable vectors are known to those of skill in the art and commercially available.

As used herein, the term “antibody” is intended to denote an immunoglobulin molecule that possesses a “variable region” antigen recognition site. The term “variable region” is intended to distinguish such domain of the immunoglobulin from domains that are broadly shared by antibodies (such as an antibody Fc domain). The variable region includes a “hypervariable region” whose residues are responsible for antigen binding. The hypervariable region includes amino acid residues from a “Complementarity Determining Region” or “CDR” (i.e., typically at approximately residues 24-34 (L1), 50-56 (L2) and 89-97 (L3) in the light chain variable domain and at approximately residues 27-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, MD. (1991)) and/or those residues from a “hypervariable loop” (i.e., residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, 1987, J. Mol. Biol. 196:901-917). “Framework Region” or “FR” residues are those variable domain residues other than the hypervariable region residues as herein defined. The term antibody includes monoclonal antibodies, multi-specific antibodies, human antibodies, humanized antibodies, synthetic antibodies, chimeric antibodies, camelized antibodies (See e.g., Muyldermans et al., 2001, Trends Biochem. Sci. 26:230; Nuttall et al., 2000, Cur. Pharm. Biotech. 1:253; Reichmann and Muyldermans, 1999, J. Immunol. Meth. 231:25; International Publication Nos. WO 94/04678 and WO 94/25591; U.S. Pat. No. 6,005,079), single-chain Fvs (scFv) (see, e.g., see Pluckthun in The Pharmacology of Monoclonal Antibodies, vol. 113, Rosenburg and Moore eds. Springer-Verlag, New York, pp. 269-315 (1994)), single chain antibodies, disulfide-linked Fvs (sdFv), intrabodies, and anti-idiotypic (anti-Id) antibodies (including, e.g., anti-Id and anti-anti-Id antibodies to antibodies). In particular, such antibodies include immunoglobulin molecules of any type (e.g., IgG, IgE, IgM, IgD, IgA and IgY), class (e.g., IgG₁, IgG₂, IgG₃, IgG₄, IgA₁ and IgA₂) or subclass.

As used herein, the term “antigen binding fragment” of an antibody refers to one or more portions of an antibody that contain the antibody's Complementarity Determining Regions (“CDRs”) and optionally the framework residues that include the antibody's “variable region” antigen recognition site, and exhibit an ability to immunospecifically bind antigen. Such fragments include Fab', F(ab′)₂, Fv, single chain (ScFv), and mutants thereof, naturally occurring variants, and fusion proteins including the antibody's “variable region” antigen recognition site and a heterologous protein (e.g., a toxin, an antigen recognition site for a different antigen, an enzyme, a receptor or receptor ligand, etc.).

As used herein, the term “fragment” refers to a peptide or polypeptide including an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, at least 150 contiguous amino acid residues, at least 175 contiguous amino acid residues, at least 200 contiguous amino acid residues, or at least 250 contiguous amino acid residues.

The term “derivative” refers to an antibody or antigen-binding fragment thereof that immunospecifically binds to the same target of a parent or reference antibody but which differs in amino acid sequence from the parent or reference antibody or antigen binding fragment thereof by including one, two, three, four, five or more amino acid substitutions, additions, deletions or modifications relative to the parent or reference antibody or antigen binding fragment thereof. In some embodiments, such derivatives will have substantially the same immunospecificity and/or characteristics, or the same immunospecificity and characteristics as the parent or reference antibody or antigen binding fragment thereof. The amino acid substitutions or additions of such derivatives can include naturally occurring (i.e., DNA-encoded) or non-naturally occurring amino acid residues. The term “derivative” encompasses, for example, chimeric or humanized variants, as well as variants having altered CH1, hinge, CH2, CH3 or CH4 regions, so as to form, for example antibodies, etc., having variant Fc regions that exhibit enhanced or impaired effector or binding characteristics.

As used herein, a “chimeric antibody” is a molecule in which different portions of the antibody are derived from different immunoglobulin molecules such as antibodies having a variable region derived from a non-human antibody and a human immunoglobulin constant region.

As used herein, the term “humanized antibody” refers to an immunoglobulin including a human framework region and one or more CDR's from a non-human (usually a mouse or rat) immunoglobulin. The non-human immunoglobulin providing the CDR's is called the “donor” and the human immunoglobulin providing the framework is called the “acceptor.” Constant regions need not be present, but if they are, they should be substantially identical to human immunoglobulin constant regions, i.e., at least about 85-99%, or about 95% or more identical. Hence, all parts of a humanized immunoglobulin, except possibly the CDR's, are substantially identical to corresponding parts of natural human immunoglobulin sequences. A humanized antibody is an antibody including a humanized light chain and a humanized heavy chain immunoglobulin. For example, a humanized antibody would not encompass a typical chimeric antibody, because, e.g., the entire variable region of a chimeric antibody is non-human.

### II. Embodiments

Prophylactic compositions for inhibiting or reducing HIV infection and methods of their use are provided. One embodiment provides an antibody or an antigen binding fragment thereof that specifically binds to an HIV protein, for example gp120, and inhibits or reduces the ability of the HIV virus to infect human cells. One embodiment provides a PGT121 antibody or antigen binding fragment thereof modified to contain a GPI membrane anchor, wherein the antibody specifically binds to an HIV protein. In some embodiments the GPI membrane anchor is in the heavy chain of the antibody or antigen fragment thereof. The membrane anchor can contain transmembrane domains, glycosylphosphatidylinositol anchors, or myristoylation motifs. In some aspects the antibody or antigen binding fragment specifically to gp120 or gp41.

One embodiment provides a PGT121 antibody or antigen binding fragment thereof wherein the antibody or antigen binding fragment is modified to include a GPI membrane anchor.

In one embodiment, the complete PGT121 Heavy Chain mRNA contains a signal sequence, heavy chain sequence, and, if included, membrane anchor sequence.

Nucleic acid and amino acid sequence for representative antibodies that can be used in preventing HIV infection are provided below. In the following nucleic acid sequences, it will be appreciated that the “T” nucleotides in the following sequences can be replaced with “U” nucleotides to generate RNA sequences.

In one embodiment the PGT121 IgG Heavy Chain Signal Sequence is encoded by a nucleic acid having 85%, 90%, 99%, or 100% sequence identity to:

(SEQ ID NO: 9)
ATGAAATGGGTCACCTTTATCAGCCTGCTGTTCCTGTTCAGCAGCGCCTA
C.

In one embodiment the PGT121 IgG Heavy Chain is encoded by a nucleic acid having 85%, 90%, 99%, or 100% sequence identity to:

(SEQ ID NO: 10)
AGCCAGATGCAGCTCCAAGAGTCTGGCCCTGGCCTGGTCAAGCCTAGCGA
AACACTGAGCCTGACCTGTTCCGTGTCCGGCGCCAGCATCAGCGATAGCT
ACTGGTCCTGGATCAGAAGAAGCCCCGGCAAAGGCCTGGAATGGATCGGC
TACGTGCACAAGAGCGGCGACACCAACTATAGCCCCAGCCTGAAGTCCAG
AGTGAACCTGAGCCTGGACACCAGCAAGAACCAGGTGTCCCTGTCTCTGG
TGGCCGCCACAGCTGCTGATAGCGGCAAGTACTACTGCGCCAGAACACTG
CACGGCAGACGGATCTATGGCATCGTGGCCTTCAACGAGTGGTTCACCTA
CTTCTACATGGACGTGTGGGGCAACGGCACCCAAGTGACAGTGTCTAGCG
CCAGCACAAAGGGCCCTAGCGTTTTCCCACTGGCTCCCAGCAGCAGAAGC
ACCAGCGAATCTACAGCCGCTCTGGGCTGCCTCGTGAAGGACTACTTTCC
TGAGCCAGTGACCGTGTCCTGGAACAGCGGCTCTCTGACATCTGGCGTGC
ACACCTTTCCAGCCGTGCTGCAAAGCAGCGGCCTGTACTCTCTGAGCAGC
GTGGTCACAGTGCCTAGCTCTAGCCTGGGCACCCAGACCTACGTGTGCAA
TGTGAATCACAAGCCCAGCAACACCAAGGTGGACAAGAGAGTGGAAATCA
AGACCTGCGGCGGAGGCAGCAAGCCTCCTACATGTCCTCCATGTCCTGCT
CCAGAAGCTGCCGGCGGACCTTCCGTGTTTCTGTTCCCTCCAAAGCCTAA
GGACACCCTGATGATCAGCAGAACCCCTGAAGTGACCTGCGTGGTGGTGG
ACGTGTCCCAAGAGGACCCCGACGTGAAGTTCAATTGGTACGTGAACGGC
GCCGAGGTGCACCACGCTCAGACAAAGCCAAGAGAGACACAGTACAACAG
CACCTACAGAGTGGTGTCCGTGCTGACCGTGACACACCAGGATTGGCTGA
ACGGCAAAGAGTACACCTGTAAAGTCTCCAACAAGGCCCTGCCTGCTCCT
ATCCAGAAAACCATCAGCAAGGACAAGGGCCAGCCTCGCGAACCCCAGGT
TTACACACTGCCTCCAAGCAGAGAGGAACTGACCAAAAATCAGGTTTCCC
TGACCTGCCTGGTTAAGGGCTTCTACCCCAGCGACATCGTGGTGGAATGG
GAGTCTAGCGGACAGCCCGAGAACACCTACAAGACCACACCTCCAGTGCT
GGACAGCGACGGCAGCTACTTCCTGTACAGCAAGCTGACAGTGGACAAGT
CCAGATGGCAGCAGGGCAACGTGTTCAGCTGCAGCGTGATGCACGAGGCC
CTGCACAACCACTACACCCAGAAGTCTCTGAGCGTCAGCCCTGGCAAG.

In one embodiment the RNA sequence for Decay Accelerating Factor GPI membrane anchor is encoded by a nucleic acid having 85%, 90%, 99%, or 100% sequence identity to:

(SEQ ID NO: 11)
CACGAGACAACCCCTAACAAAGGCAGCGGCACCACCTCTGGCACCACAAG
ACTGCTGTCTGGCCACACCTGTTTCACACTGACCGGCCTGCTGGGCACAC
TGGTTACAATGGGACTGCTGACC.

The complete PGT121 Light Chain mRNA consists of a signal sequence and light chain sequence.

In one embodiment the RNA sequence for IgG Light Chain Signal Sequence is encoded by a nucleic acid having 85%, 90%, 99%, or 100% sequence identity to

(SEQ ID NO: 12)
ATGAAGTGGGTGACCTTCATCAGCCTGCTGTTCCTGTTCAGCAGCGCCTA
C.

In one embodiment, the RNA sequence for PGT121 IgG Light Chain is encoded by a nucleic acid having 85%, 90%, 99%, or 100% sequence identity to:

(SEQ ID NO: 13)
AGCAGCGACATCAGCGTGGCCCCCGGCGAGACCGCCAGAATCAGCTGCGG
CGAGAAGAGCCTGGGCAGCAGAGCCGTGCAGTGGTACCAGCACAGAGCCG
GCCAGGCCCCCAGCCTGATCATCTACAACAACCAGGACAGACCCAGCGGC
ATCCCCGAGAGATTCAGCGGCAGCCCCGACAGCCCCTTCGGCACCACCGC
CACCCTGACCATCACCAGCGTGGAGGCCGGCGACGAGGCCGACTACTACT
GCCACATCTGGGACAGCAGAGTGCCCACCAAGTGGGTGTTCGGCGGCGGC
ACCACCCTCACCGTGCTGAGAGCCGTGGCCGCCCCCAGCGTGTTCATCTT
CCCCCCCAGCGAGGACCAGGTGAAGAGCGGCACCGTGAGCGTGGTGTGCC
TGCTGAACAACTTCTACCCCAGAGAGGCCAGCGTGAAGTGGAAGGTGGAC
GGCGTGCTGAAGACCGGCAACAGCCAGGAGAGCGTGACCGAGCAGGACAG
CAAGGACAACACCTACAGCCTGAGCAGCACCCTGACCCTGAGCAGCACCG
ACTACCAGAGCCACAACGTGTACGCCTGCGAGGTGACCCACCAGGGCCTG
AGCAGCCCCGTGACCAAGAGCTTCAACAGAGGCGAGTGC.

One embodiment provides a vector comprising SEQ ID Nos 9-13. In one embodiment the vector is an mRNA.

One embodiment provides the complete 10E8.4 iMab containing of four distinct mRNA sequences—2 different heavy chain sequences and 2 different light chain sequences. Both complete Heavy Chain mRNAs consist of a signal sequence, heavy chain sequence, and, if included, membrane anchor sequence.

In one embodiment the RNA sequence for the 10E8.4/iMab 10E8.4 IgG Heavy Chain Signal Sequence is encoded by a nucleic acid having 85%, 90%, 99%, or 100% sequence identity to:

(SEQ ID NO: 14)
ATGGGATGGTCCTGCATCATCCTGTTCCTGGTGGCCACCGCCACCGGCGT
GCACAGC.

In one embodiment the RNA sequence for the 10E8.4/iMab 10E8.4 IgG Heavy Chain Sequence is encoded by a nucleic acid having 85%, 90%, 99%, or 100% sequence identity to:

(SEQ ID NO: 15)
GAGGTGCGCCTCGTGGAGAGCGGCGGAGGCCTGGTGAAGCCGGGCGGCAG
CCTGCGGCTGAGCTGTAGCGCCAGCGGCTTTAACTTCGACGACGCCTGGA
TGACATGGGTGCGGCAGCCTCCTGGCAAGGGCCTGGAATGGGTCGGCAGA
ATCAGTGGCCCTGGCGAGGGCTGGTCTGTGGATTACGCTGAGTCTGTGAA
GGGCAGATTCACCATCAGCAGAAAGAATAGCAAGAACACACTGTACCTGG
AAATGAACAACGTGCGGACCGAGGATACCGGATATTACTTCTGCGCCAGA
ACCGGCAAGCACTACGACTTCTGGAGCGGCTACCCTCCTGGGGAGGAATA
CTTTCAGGACTGGGGCCAAGGCACAAAGGTGATCGTGAGCAGCGCCTCTA
CCAAAGGCCCCTCCGTGTTCCCACTGGCTCCTAGCTCTAAGAGCACCAGC
GGCGGCACAGCCGCTCTTGGATGTCTGGTCAAGGACTACTTCCCTGAGCC
TGTTACAGTGTCCTGGAACAGCGGGGCCCTGACCAGCGGCGTGCACACCT
TCCCCGCCGTACTGCAGAGCAGTGGACTGTATAGCCTCTCCAGCGTTGTG
ACCGTGCCCAGCTCCAGCCTGGGCACCCAGACCTACATCTGCAATGTGAA
CCACAAGCCATCTAACACCAAGGTGGACAAGAAGGTGGAACCCAAATCTT
GCGACAAGACCCACACATGTCCTCCTTGCCCTGCCCCTGAGTTCGAGGGC
GGACCTTCTGTGTTTCTGTTTCCACCTAAGCCTAAGGACACCCTGATGAT
CTCTAGAACCCCTGAGGTGACCTGCGTGGTGGTGGACGTGTCTCACGAGG
ACCCCGAGGTGAAATTCAACTGGTACGTGGATGGCGTGGAAGTGCACAAC
GCCAAGACAAAACCCAGAGAGGAGCAGTACAACAGCACATACCGGGTGGT
GTCCGTCTTGACCGTGCTGCACCAGGATTGGCTGAACGGCAAGGAATACA
AGTGCAAGGTCAGCAATAAGGCCCTGCCTGCTAGCATCGAGAAGACCATT
TCTAAAGCCAAGGGCCAACCTCGGGAACCCCAGGTGTACACCCTGCCTCC
ATGTAGAGATGAACTGACAAAGAACCAGGTGTCCCTGTGGTGCCTGGTGA
AGGGCTTCTACCCCTCTGATATCGCCGTGGAATGGGAGAGCAATGGCCAG
CCCGAAAACAATTACAAAACCACCCCTCCAGTGCTGGACAGCGACGGCAG
CTTCTTCCTGTACAGCAAGCTGACCGTGGACAAGAGCAGATGGCAGCAGG
GCAACGTGTTCAGCTGCAGCGTGCTGCATGAGGCCCTGCACAGCCACTAT
ACCCAGAAATCTCTGAGCCTGAGCCCCGGAAAA.

In one embodiment, the RNA sequence for the 10E8.4/iMab MV1 IgG Heavy Chain Signal Sequence is encoded by a nucleic acid having 85%, 90%, 99%, or 100% sequence identity to:

(SEQ ID NO: 16)
ATGGGATGGTCCTGCATCATCCTGTTCCTGGTGGCCACCGCCACCGGCG
TGCACAGC.

In one embodiment the RNA sequence for the 10E8.4/iMab MV1 IgG Heavy Chain Sequence is encoded by a nucleic acid having 85%, 90%, 99%, or 100% sequence identity to:

(SEQ ID NO: 17)
CAGGTGCAGCTGCAACAGAGCGGCCCCGAAGTGGTCAAACCAGGCGCCA
GCGTGAAAATGAGCTGTAAGGCTAGTGGATACACCTTTACCAGCTACGT
GATCCACTGGGTGCGGCAGAAACCTGGCCAGGGCCTGGACTGGATCGGC
TACATCAACCCCTACAACGACGGCACTGATTACGACGAGAAGTTCAAGG
GCAAGGCCACACTGACCTCCGACACAAGCACATCCACCGCTTATATGGA
ACTGAGCAGCCTGAGAAGCGAGGACACCGCAGTGTACTACTGCGCCAGA
GAAAAAGATAACTACGCCACCGGAGCCTGGTTCGCCTACTGGGGCCAGG
GCACACTGGTTACCGTCTCTAGCGCCAGCACCGCCGCCCCTAGCGTTTT
CATCTTCCCTCCTAGCGACGAGCAACTGAAGAGCGGCACCGCTAGCGTC
GTGTGCCTGCTGAACAACTTCTACCCTCGGGAAGCCAAGGTCCAGTGGA
AGGTGGACAACGCCCTGCAGAGCGGAAATAGCCAGGAGTCTGTGACGGA
ACAAGATAGCAAGGACAGCACATACTCCCTGAGCAGCACACTGACCCTG
AGCAAGGCCGATTACGAGAAGCACAAGGTGTATGCCTGTGAAGTGACCC
ACCAGGGCCTGTCTTCTCCTGTGACCAAGAGCTTCAATAGAGGCGAGTG
CGACAAGACCCACACCTGTCCACCGTGCCCCGCTCCCGAGTTCGAGGGC
GGCCCTAGCGTGTTTCTGTTTCCACCTAAGCCCAAGGATACCCTGATGA
TCTCTAGAACACCTGAGGTGACATGTGTGGTGGTGGACGTGAGCCACGA
GGATCCTGAGGTGAAATTCAACTGGTACGTGGATGGCGTGGAAGTGCAC
AACGCCAAAACAAAACCCAGAGAGGAGCAGTACAATTCTACCTACCGGG
TGGTGTCCGTGCTGACCGTGCTGCACCAGGACTGGCTGAACGGCAAAGA
GTACAAGTGCAAGGTGTCCAACAAGGCCCTGCCTGCCTCTATCGAGAAG
ACCATCAGCAAGGCTAAGGGACAACCTAGAGAACCTCAGGTTTGCACCC
TGCCTCCTTCCCGCGACGAACTGACCAAGAACCAGGTGAGTCTGTCTTG
CGCCGTGAAGGGCTTCTACCCCAGCGACATCGCTGTGGAATGGGAGAGC
AACGGCCAGCCCGAGAACAACTATAAGACAACCCCTCCTGTGCTGGATA
GCGACGGCAGCTTTTTCCTGGTGAGCAAGCTGACCGTGGACAAGAGCAG
ATGGCAGCAGGGCAACGTGTTCAGCTGCAGCGTGCTGCATGAGGCCCTG
CACAGCCACTATACCCAGAAATCTCTGAGCCTGAGCCCCGGAAAA.

In one embodiment the RNA sequence for Decay Accelerating Factor GPI membrane anchor (if included) is encoded by a nucleic acid sequence having 85%, 90%, 99%, or 100% sequence identity to:

(SEQ ID NO: 18)
CATGAGACAACCCCTAACAAGGGCTCTGGAACAACAAGCGGAACAACCA
GACTGCTGAGCGGCCACACATGCTTCACCCTGACCGGCCTGCTGGGAAC
ACTGGTGACCATGGGCCTGCTGACT.

One embodiment provides a vector containing SEQ ID Nos:14-22. In one embodiment the vector is mRNA.

In one embodiment, both complete Light Chain mRNAs contain a signal sequence and heavy chain sequence.

In one embodiment the RNA sequence for the 10E8.4/iMab 10E8.4 IgG Light Chain Signal Sequence is encoded by a nucleic acid sequence having 85%, 90%, 99%, or 100% sequence identity to:

(SEQ ID NO: 19)
ATGGGCTGGTCCTGCATCATCCTGTTCCTGGTGGCCACCGCCACAGGCA
GC.

In one embodiment, the RNA sequence for the 10E8.4/iMab 10E8.4 IgG Light Chain Sequence is encoded by a nucleic acid sequence having 85%, 90%, 99%, or 100% sequence identity to:

(SEQ ID NO: 20)
TGGGCCGCTTCTGAGCTGACACAGGACCCCGCCGTGTCCGTGGCCCTGA
AGCAAACAGTGACCATCACTTGTAGAGGCGACAGCCTGAGAAGCCACTA
CGCCAGCTGGTACCAGAAAAAGACCGGCCAGGCCCCTAAGCTGCTGTTC
TACGGCAAGAACAATCGCCCCAGCGGAGTGCCCGACAGATTCAGCGGAA
GCGCCTCTGGCAACCGGGCCTCTCTGACCATCAGCGGAGCCCAGGCTGA
AGATGACGCCGAGTACTACTGCAGCAGCAGAGATAAGTCCGGCTCTAGA
CTGAGCGTGTTCGGCGGCGGAACAAAGCTGACCGTGCTGAGCCAACCTA
AGGCTGCCCCTAGCGTGACCCTGTTTCCACCTAGCTCCGAGGAACTCCA
GGCCAACAAGGCCACCCTGGTCTGCCTGATCAGCGACTTCTATCCTGGC
GCCGTGACCGTCGCCTGGAAAGCTGATAGCAGCCCTGTGAAGGCCGGCG
TGGAAACCACCACCCCTTCAAAGCAGAGCAACAACAAATACGCCGCTTC
TTCTTACCTGTCTCTGACCCCCGAGCAGTGGAAGTCCCACCGGAGCTAC
AGCTGCCAGGTGACACACGAGGGCAGCACCGTGGAAAAGACAGTTGCAC
CAACAGAGTGTAGC.

In one embodiment, the RNA sequence for the 10E8.4/iMab MV1 IgG Light Chain Signal Sequence is encoded by a nucleic acid sequence 85%, 90%, 99%, or 100% sequence identity to:

(SEQ ID NO: 21)
ATGGGCTGGTCGTGCATCATCCTGTTTCTGGTGGCCACAGCTACAGGCT
CT.

In one embodiment the RNA sequence for the 10E8.4/iMab MV1 IgG Light Chain Sequence is encoded by a nucleic acid having 85%, 90%, 99%, or 100% sequence identity to:

(SEQ ID NO: 22)
TGGGCCGACATTGTGATGACCCAAAGCCCTGACAGCCTGGCTGTGTCTC
TCGGCGAACGGGTGACAATGAACTGCAAGAGCAGCCAGTCCCTGCTGTA
TAGCACCAACCAGAAAAACTACCTGGCCTGGTATCAGCAGAAGCCCGGC
CAGTCTCCTAAGCTGCTGATCTACTGGGCTTCTACCAGAGAGAGCGGTG
TTCCTGATAGATTCAGCGGCAGCGGCAGCGGCACTGATTTCACCCTGAC
CATCAGCAGCGTGCAGGCCGAGGACGTGGCCGTGTACTACTGCCAGCAA
TACTACAGCTACAGAACCTTCGGCGGAGGCACAAAACTGGAAATCAAGA
GCTCCGCCAGCACCAAGGGCCCTTCTGTGTTCCCACTGGCCCCAAGCTC
TAAGTCCACCTCCGGAGGAACCGCCGCCCTGGGCTGTCTGGTCAAGGAC
TACTTCCCTGAGCCTGTGACAGTGTCCTGGAATAGCGGCGCCCTGACCA
GCGGAGTGCACACCTTTCCCGCCGTGCTGCAGAGCTCAGGCCTGTACAG
CCTTTCTAGCGTGGTGACAGTGCCCAGCAGCAGCCTGGGCACACAGACC
TACATCTGCAACGTGAACCACAAGCCTAGCAATACCAAAGTGGACAAGA
AGGTCGAGCCCAAGTCTTGT.

In one embodiment, the complete J3 VHH mRNA consists of a signal sequence and VHH sequence.

In one embodiment, the RNA sequence for the J3 VHH Signal Sequence is encoded by a nucleic acid sequence having 85%, 90%, 99%, or 100% sequence identity to:

(SEQ ID NO: 23)
ATGAAATGGGTCACATTCATCTCTCTGCTGTTCCTGTTCAGCAGCGCCT
ATAGC.

In one embodiment the RNA sequence for the J3 VHH Sequence is encoded by a nucleic acid having 85%, 90%, 99%, or 100% sequence identity to:

(SEQ ID NO: 24)
GAGGTGCAGCTGGTGGAAAGCGGCGGAGGCCTCGTGCAAGCTGGAGGAT
TTCTGAGACTGAGCTGCGAGCTGCGGGGCAGCATCTTCAACCAGTACGC
CATGGCCTGGTTCCGGCAGGCCCCTGGCAAGGAACGGGAATTCGTGGCC
GGCATGGGAGCTGTGCCCCACTACGGCGAGTTCGTTAAAGGCAGATTCA
CCATCTCTAGAGATAATGCCAAGTCCACCGTGTACCTGCAGATGAGCAG
CCTGAAGCCTGAGGACACCGCCATCTACTTCTGCGCCAGAAGCAAGAGC
ACATACATCAGCTACAACAGCAACGGCTACGACTACTGGGGCCGCGGCA
CACAGGTGACCGTGTCCAGC.

In one embodiment, the RNA sequence for the Decay Accelerating Factor GPI membrane anchor (if included) is encoded by a nucleic acid sequence 85%, 90%, 99%, or 100% sequence identity to:

(SEQ ID NO: 25)
GGCGGCGGCGGCTCTCACGAGACAACCCCAAACAAGGGTTCCGGCACCA
CCAGCGGAACCACCAGACTGCTGTCTGGCCACACCTGTTTTACCCTGAC
AGGCCTGCTGGGCACCCTGGTGACAATGGGCCTGCTGACA.

One embodiment provides a vector containing SEQ ID Nos:23-24. In one embodiment the vector is mRNA.

### Protein Sequences

In one embodiment the protein sequence for PGT121 IgG Heavy Chain Signal Sequence has 85%, 90%, 99%, or 100% sequence identity to:

(SEQ ID NO: 26)
MKWVTFISLLFLFSSAY.

In one embodiment the protein sequence for PGT121 IgG Heavy Chain 85%, 90%, 99%, or 100% sequence identity to:

(SEQ ID NO: 27)
SQMQLQESGPGLVKPSETLSLTCSVSGASISDSYWSWIRRSPGKGLEWI
GYVHKSGDTNYSPSLKSRVNLSLDTSKNQVSLSLVAATAADSGKYYCAR
TLHGRRIYGIVAFNEWFTYFYMDVWGNGTQVTVSSASTKGPSVFPLAPS
SRSTSESTAALGCLVKDYFPEPVTVSWNSGSLTSGVHTFPAVLQSSGLY
SLSSVVTVPSSSLGTQTYVCNVNHKPSNTKVDKRVEIKTCGGGSKPPTC
PPCPAPEAAGGPSVFLFPPKPKDTLMISRTPEVTCVVVDVSQEDPDVKF
NWYVNGAEVHHAQTKPRETQYNSTYRVVSVLTVTHQDWLNGKEYTCKVS
NKALPAPIQKTISKDKGQPREPQVYTLPPSREELTKNQVSLTCLVKGFY
PSDIVVEWESSGQPENTYKTTPPVLDSDGSYFLYSKLTVDKSRWQQGNV
FSCSVMHEALHNHYTQKSLSVSPGK.

In one embodiment, the protein sequence for Decay Accelerating Factor GPI membrane anchor (if included) has 85%, 90%, 99%, or 100% sequence identity to:

(SEQ ID NO: 28)
HETTPNKGSGTTSGTTRLLSGHTCFTLTGLLGTLVTMGLLT.

In one embodiment, the protein sequence for PGT121 IgG Light Chain Signal Sequence has 85%, 90%, 99%, or 100% sequence identity to:

(SEQ ID NO: 29)
MKWVTFISLLFLFSSAY.

In one embodiment the protein sequence for PGT121 IgG Light Chain Sequence has 85%, 90%, 99%, or 100% sequence identity to:

(SEQ ID NO: 30)
SSDISVAPGETARISCGEKSLGSRAVQWYQHRAGQAPSLIIYNNQDRPS
GIPERFSGSPDSPFGTTATLTITSVEAGDEADYYCHIWDSRVPTKWVFG
GGTTLTVLRAVAAPSVFIFPPSEDQVKSGTVSVVCLLNNFYPREASVKW
KVDGVLKTGNSQESVTEQDSKDNTYSLSSTLTLSSTDYQSHNVYACEVT
HQGLSSPVTKSFNRGEC.

One embodiment provides an antibody or an antigen binding fragment thereof comprising a heavy chain amino acid sequence according to SEQ ID NO:27, a GPI membrane anchor have the amino acid sequence according to SEQ ID NO:28, and a light chain amino acid sequence according to SEQ ID NO:30.

In one embodiment, the protein sequence for the 10E8.4/iMab 10E8.4 IgG Heavy Chain Signal Sequence has 85%, 90%, 99%, or 100% sequence identity to:

(SEQ ID NO: 31)
MGWSCIILFLVATATGVHS.

In one embodiment the protein sequence for the 10E8.4/iMab 10E8.4 IgG Heavy Chain Sequence has 85%, 90%, 99%, or 100% sequence identity to:

(SEQ ID NO: 32)
EVRLVESGGGLVKPGGSLRLSCSASGFNFDDAWMTWVRQPPGKGLEWVG
RISGPGEGWSVDYAESVKGRFTISRKNSKNTLYLEMNNVRTEDTGYYFC
ARTGKHYDFWSGYPPGEEYFQDWGQGTKVIVSSASTKGPSVFPLAPSSK
STSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPA
PEFEGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALP
ASIEKTISKAKGQPREPQVYTLPPCRDELTKNQVSLWCLVKGFYPSDIA
VEWESNGQPENNYKTTPPVLDSDGSFFLYSKLTVDKSRWQQGNVFSCSV
LHEALHSHYTQKSLSLSPGK.

In one embodiment, the protein sequence for the 10E8.4/iMab MV1 IgG Heavy Chain Signal Sequence has 85%, 90%, 99%, or 100% sequence identity to:

(SEQ ID NO: 33)
MGWSCIILFLVATATGVHS.

In one embodiment, the protein sequence for the 10E8.4/iMab MV1 IgG Heavy Chain Sequence has 85%, 90%, 99%, or 100% sequence identity to:

(SEQ ID NO: 34)
QVQLQQSGPEVVKPGASVKMSCKASGYTFTSYVIHWVRQKPGQGLDWIG
YINPYNDGTDYDEKFKGKATLTSDTSTSTAYMELSSLRSEDTAVYYCAR
EKDNYATGAWFAYWGQGTLVTVSSASTAAPSVFIFPPSDEQLKSGTASV
VCLLNNFYPREAKVQWKVDNALQSGNSQESVTEQDSKDSTYSLSSTLTL
SKADYEKHKVYACEVTHQGLSSPVTKSFNRGECDKTHTCPPCPAPEFEG
GPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPASIEK
TISKAKGQPREPQVCTLPPSRDELTKNQVSLSCAVKGFYPSDIAVEWES
NGQPENNYKTTPPVLDSDGSFFLVSKLTVDKSRWQQGNVFSCSVLHEAL
HSHYTQKSLSLSPGK.

In one embodiment, the protein sequence for Decay Accelerating Factor GPI membrane anchor (if included) has 85%, 90%, 99%, or 100% sequence identity to:

(SEQ ID NO: 35)
HETTPNKGSGTTSGTTRLLSGHTCFTLTGLLGTLVTMGLLT.

In one embodiment, the protein sequence for the 10E8.4/iMab 10E8.4 IgG Light Chain Signal Sequence has 85%, 90%, 99%, or 100% sequence identity to:

(SEQ ID NO: 36)
MGWSCIILFLVATATGS.

In one embodiment the protein sequence for the 10E8.4/iMab 10E8.4 IgG Light Chain Sequence has 85%, 90%, 99%, or 100% sequence identity to:

(SEQ ID NO: 37)
WAASELTQDPAVSVALKQTVTITCRGDSLRSHYASWYQKKTGQAPKLLF
YGKNNRPSGVPDRFSGSASGNRASLTISGAQAEDDAEYYCSSRDKSGSR
LSVFGGGTKLTVLSQPKAAPSVTLFPPSSEELQANKATLVCLISDFYPG
AVTVAWKADSSPVKAGVETTTPSKQSNNKYAASSYLSLTPEQWKSHRSY
SCQVTHEGSTVEKTVAPTECS.

In one embodiment the protein sequence for the 10E8.4/iMab MV1 IgG Light Chain Signal Sequence has 85%, 90%, 99%, or 100% sequence identity to:

(SEQ ID NO: 38)
MGWSCIILFLVATATGS.

In one embodiment the protein sequence for the 10E8.4/iMab MV1 IgG Light Chain Sequence has 85%, 90%, 99%, or 100% sequence identity to:

(SEQ ID NO: 39)
WADIVMTQSPDSLAVSLGERVTMNCKSSQSLLYSTNQKNYLAWYQQKPG
QSPKLLIYWASTRESGVPDRFSGSGSGTDFTLTISSVQAEDVAVYYCQQ
YYSYRTFGGGTKLEIKSSASTKGPSVFPLAPSSKSTSGGTAALGCLVKD
YFPEPVTVSWNSGALTSGVHTFPAVLQSSGLYSLSSVVTVPSSSLGTQT
YICNVNHKPSNTKVDKKVEPKSC.

One embodiment provides an antibody or antigen binding fragment thereof having a first heavy chain with an amino acid sequence according to SEQ ID NO:32, a second heavy chain having an amino acid sequence according to SEQ ID NO:34, a GPI membrane anchor with an animo acid sequence according to 35, a first light chain with an amino acid sequence according to 37, and a second light chain having an amino acid sequence according to SEQ ID NO:39.

In one embodiment the protein sequence for the J3 VHH Signal Sequence has 85%, 90%, 99%, or 100% sequence identity to:

(SEQ ID NO: 40)
MKWVTFISLLFLFSSAYS.

In one embodiment the protein sequence for the J3 VHH Sequence has 85%, 90%, 99%, or 100% sequence identity to:

(SEQ ID NO: 41)
EVQLVESGGGLVQAGGFLRLSCELRGSIFNQYAMAWFRQAPGKEREFVA
GMGAVPHYGEFVKGRFTISRDNAKSTVYLQMSSLKPEDTAIYFCARSKS
TYISYNSNGYDYWGRGTQVTVSS.

In one embodiment, the protein sequence for the Decay Accelerating Factor GPI membrane anchor (if included) has 85%, 90%, 99%, or 100% sequence identity to:

(SEQ ID NO: 42)
GGGGSHETTPNKGSGTTSGTTRLLSGHTCFTLTGLLGTLVTMGLLT.

One embodiment provides an antibody or an antigen binding fragment thereof having a heavy chain with an amino acid sequence according to SEQ ID NO:41 and a GPI membrane anchor according to SEQ ID NO:42.

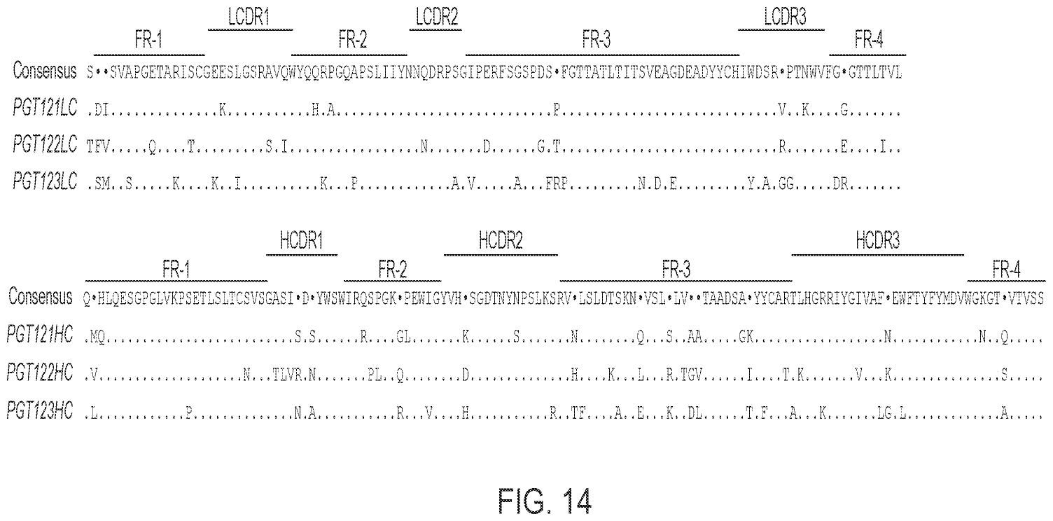

Another embodiment provides a recombinant genetic construct that encodes an antibody or an antigen binding fragment thereof that specifically binds to an HIV protein, for example gp120, and inhibits or reduces the ability of the HIV virus to infect human cells wherein the antibody is modified to include a GPI membrane anchor. In one embodiment, the antibody is a PGT121 antibody as shown in FIG. 14, wherein the antibody is modified to include a GPI anchor, for example in the heavy chain of the antibody. In some embodiments, the genetic construct is an RNA construct, for example a mRNA construct. In some embodiments, the mRNA construct is fully modified with complete substitution of uridine with n1-methyl-pseudouridine.

Another embodiment provides a pharmaceutical composition consisting of the mRNA construct that encodes an antibody or an antigen binding fragment thereof that specifically binds to an HIV protein, for example gp120, and inhibits or reduces the ability of the HIV virus to infect human cells wherein the antibody is modified to include a GPI membrane anchor and water. In some embodiments, the pharmaceutical composition also contains a buffer.

One embodiment provides a method for prophylactically inhibiting or reducing HIV infection of a female subject comprising the steps of transfecting cervicovaginal epithelial cells in the subject with the construct encoding a PGT121 antibody or antigen binding fragment thereof wherein the antibody or antigen binding fragment is modified to include a GPI membrane anchor. In some embodiments, the construct is an mRNA construct.

Another embodiment provides a method for prophylactically inhibiting or reducing HIV infection in a female subject comprising the step of administering the pharmaceutical composition consisting of the mRNA construct encoding a that encodes an antibody or an antigen binding fragment thereof that specifically binds to an HIV protein, for example gp120, and inhibits or reduces the ability of the HIV virus to infect human cells wherein the antibody is modified to include a GPI membrane anchor and water.

Another embodiment provides a kit comprising a container comprising the that encodes an antibody or an antigen binding fragment thereof that specifically binds to an HIV protein, for example gp120, and inhibits or reduces the ability of the HIV virus to infect human cells wherein the antibody is modified to include a GPI membrane anchor and an aerosol delivery device. In one embodiment, the antibody is a PGT121 antibody as shown in FIG. 14, wherein the antibody is modified to include a GPI anchor, for example in the heavy chain of the antibody. In some embodiments, the genetic construct is an RNA construct, for example a mRNA construct. In some embodiments, the mRNA construct is fully modified with complete substitution of uridine with n1-methyl-pseudouridine. In some embodiments the delivery device is an atomizer or a dual-chamber syringe containing lyophilized mRNA and water and an atomizer suitable for self-insertion into the FRT. Exemplary atomizers include but are not limited to a Penn Century microsprayer (20 um), Teleflex atomizer (30-100 um), an impinging jet atomizer (5-10 um), a pediatric nebulizer (5-7 um), and a droplet stream generator. The atomizers can be used to vary both droplet velocity and size.

One embodiment provides an antibody comprising a heavy chain encoded by a nucleic acid having 85%, 90%, 95%, 99%, or 100% sequence identity to SEQ ID NO:1 and a light chain encoded by nucleic acid having 85%, 90%, 95%, 99%, or 100% sequence identity to SEQ ID NO:2.

### PGT121 NanoLuc® Geneblock LC-mRNA

(SEQ ID NO: 1)
CGATTGGCGGAAGGCCGTCAAGGCCGCATTTTTAAGCTTTAATACGAC
TCACTATAGGGAAATAAGAGAGAAAAGAAGAGTAAGAAGAAATATA
AGAGCCACCATGAAGTGGGTGACCTTCATCAGCCTGCTGTTCCTGTTC
AGCAGCGCCTACAGCAGCGACATCAGCGTGGCCCCCGGCGAGACCGC
CAGAATCAGCTGCGGCGAGAAGAGCCTGGGCAGCAGAGCCGTGCAGT
GGTACCAGCACAGAGCCGGCCAGGCCCCCAGCCTGATCATCTACAAC
AACCAGGACAGACCCAGCGGCATCCCCGAGAGATTCAGCGGCAGCCC
CGACAGCCCCTTCGGCACCACCGCCACCCTGACCATCACCAGCGTGGA
GGCCGGCGACGAGGCCGACTACTACTGCCACATCTGGGACAGCAGAG
TGCCCACCAAGTGGGTGTTCGGCGGCGGCACCACCCTCACCGTGCTGA
GAGCCGTGGCCGCCCCCAGCGTGTTCATCTTCCCCCCCAGCGAGGACC
AGGTGAAGAGCGGCACCGTGAGCGTGGTGTGCCTGCTGAACAACTTC
TACCCCAGAGAGGCCAGCGTGAAGTGGAAGGTGGACGGCGTGCTGAA
GACCGGCAACAGCCAGGAGAGCGTGACCGAGCAGGACAGCAAGGAC
AACACCTACAGCCTGAGCAGCACCCTGACCCTGAGCAGCACCGACTA
CCAGAGCCACAACGTGTACGCCTGCGAGGTGACCCACCAGGGCCTGA
GCAGCCCCGTGACCAAGAGCTTCAACAGAGGCGAGTGCGGAGGCGGG
GGCAGCGTCTTCACACTCGAAGATTTCGTTGGGGACTGGCGACAGAC
AGCCGGCTACAACCTGGACCAAGTCCTTGAACAGGGAGGTGTGTCCA
GTTTGTTTCAGAATCTCGGGGTGTCCGTAACTCCGATCCAAAGGATTG
TCCTGAGCGGTGAAAATGGGCTGAAGATCGACATCCATGTCATCATCC
CGTATGAAGGTCTGAGCGGCGACCAAATGGGCCAGATCGAAAAAATT
TTTAAGGTGGTGTACCCTGTGGATGATCATCACTTTAAGGTGATCCTG
CACTATGGCACACTGGTAATCGACGGGGTTACGCCGAACATGATCGA
CTATTTCGGACGGCCGTATGAAGGCATCGCCGTGTTCGACGGCAAAA
AGATCACTGTAACAGGGACCCTGTGGAACGGCAACAAAATTATCGAC
GAGCGCCTGATCAACCCCGACGGCTCCCTGCTGTTCCGAGTAACCATC
AACGGAGTGACCGGCTGGCGGCTGTGCGAACGCATTCTGGCGTGATA
AGCTGCCTTCTGCGGGGCTTGCCTTCTGGCCATGCCCTTCTTCTCTCC
CTTGCACCTGTACCTCTTGGTCTTTGAATAAAGCCTGAGTAGGAAGGC
GGCCGCAAAAACTGGGCC

### DAF GPI Anchored PGT121 HC mRNA

(SEQ ID NO: 2)
TTTTAAGCTTTAATACGACTCACTATAGGGAAATAAGAGAGAAAAGA
AGAGTAAGAAGAAATATAAGAGCCACCATGAAATGGGTCACCTTTAT
CAGCCTGCTGTTCCTGTTCAGCAGCGCCTACAGCCAGATGCAGCTCCA
AGAGTCTGGCCCTGGCCTGGTCAAGCCTAGCGAAACACTGAGCCTGA
CCTGTTCCGTGTCCGGCGCCAGCATCAGCGATAGCTACTGGTCCTGGA
TCAGAAGAAGCCCCGGCAAAGGCCTGGAATGGATCGGCTACGTGCAC
AAGAGCGGCGACACCAACTATAGCCCCAGCCTGAAGTCCAGAGTGAA
CCTGAGCCTGGACACCAGCAAGAACCAGGTGTCCCTGTCTCTGGTGGC
CGCCACAGCTGCTGATAGCGGCAAGTACTACTGCGCCAGAACACTGC
ACGGCAGACGGATCTATGGCATCGTGGCCTTCAACGAGTGGTTCACCT
ACTTCTACATGGACGTGTGGGGCAACGGCACCCAAGTGACAGTGTCT
AGCGCCAGCACAAAGGGCCCTAGCGTTTTCCCACTGGCTCCCAGCAGC
AGAAGCACCAGCGAATCTACAGCCGCTCTGGGCTGCCTCGTGAAGGA
CTACTTTCCTGAGCCAGTGACCGTGTCCTGGAACAGCGGCTCTCTGAC
ATCTGGCGTGCACACCTTTCCAGCCGTGCTGCAAAGCAGCGGCCTGTA
CTCTCTGAGCAGCGTGGTCACAGTGCCTAGCTCTAGCCTGGGCACCCA
GACCTACGTGTGCAATGTGAATCACAAGCCCAGCAACACCAAGGTGG
ACAAGAGAGTGGAAATCAAGACCTGCGGCGGAGGCAGCAAGCCTCCT
ACATGTCCTCCATGTCCTGCTCCAGAAGCTGCCGGCGGACCTTCCGTG
TTTCTGTTCCCTCCAAAGCCTAAGGACACCCTGATGATCAGCAGAACC
CCTGAAGTGACCTGCGTGGTGGTGGACGTGTCCCAAGAGGACCCCGA
CGTGAAGTTCAATTGGTACGTGAACGGCGCCGAGGTGCACCACGCTC
AGACAAAGCCAAGAGAGACACAGTACAACAGCACCTACAGAGTGGT
GTCCGTGCTGACCGTGACACACCAGGATTGGCTGAACGGCAAAGAGT
ACACCTGTAAAGTCTCCAACAAGGCCCTGCCTGCTCCTATCCAGAAAA
CCATCAGCAAGGACAAGGGCCAGCCTCGCGAACCCCAGGTTTACACA
CTGCCTCCAAGCAGAGAGGAACTGACCAAAAATCAGGTTTCCCTGAC
CTGCCTGGTTAAGGGCTTCTACCCCAGCGACATCGTGGTGGAATGGGA
GTCTAGCGGACAGCCCGAGAACACCTACAAGACCACACCTCCAGTGC
TGGACAGCGACGGCAGCTACTTCCTGTACAGCAAGCTGACAGTGGAC
AAGTCCAGATGGCAGCAGGGCAACGTGTTCAGCTGCAGCGTGATGCA
CGAGGCCCTGCACAACCACTACACCCAGAAGTCTCTGAGCGTCAGCC
CTGGCAAGCACGAGACAACCCCTAACAAAGGCAGCGGCACCACCTCT
GGCACCACAAGACTGCTGTCTGGCCACACCTGTTTCACACTGACCGGC
CTGCTGGGCACACTGGTTACAATGGGACTGCTGACCTGATAAGCTGCC
TTCTGCGGGGCTTGCCTTCTGGCCATGCCCTTCTTCTCTCCCTTGCAC
CTGTACCTCTTGGTCTTTGAATAAAGCCTGAGTAGGAAGGCGGCCGCA
A AAA

### III. Discussion

The vaginal mucosa has been explored to deliver drugs both locally and systemically due to its large surface area, a high degree of vascularization, avoidance of first-pass metabolism by the liver, good drug permeability, and its accessibility to allow self-application. Though several approaches have had successes, protection from HIV acquisition has been a challenge, since most antimicrobials also damage the epithelium, creating inflammatory conditions and a portal of entry for the virus.

With the aerosolized, unformulated mRNA transfection platform described here, the local production of transgene protein by the native epithelium ensures that high tissue and secretion concentrations are achieved quickly, while also ensuring host glycosylation and other post-translational motifs are preserved. The entire lower FRT was conducive to transfection by aerosolized, naked, synthetic mRNA. Tethering PGT121 to the transfected cell surface using a GPI anchor promoted genital secretion and tissue concentrations well above neutralizing concentrations at 28 days post-transfection. Moreover, mRNA-expressed aPGT121 retained the neutralizing capacity and effector-cell function of parental PGT121. Ex vivo challenge of rhesus macaque FRT biopsy explants with SHIV demonstrated tissue level protection due to aPGT121 tissue expression, as well as antibody neutralization activity in secretions.

Recent data using fluorescent HIV suggest that all areas of the female reproductive tract can be considered vulnerable to infection. Thus, prophylactic approaches will likely need to protect the entirety of the lower FRT. The data described herein including IVIS imaging at 1, 14, and 28 days demonstrated that the entirety of the lower FRT epithelium expressed aPGT121. However, by 14 and 28 days, the cervix was the site of the majority of luminescent signal, perhaps in part due to differential turnover and shedding of epithelial cells, differences in cellular metabolism, and the more superficial nature of basal stem cells at the cervix compared to the stratified vagina. Surprisingly, at 28 days post-transfection, tissue lysates from each portion of the FRT (caudal vagina, rostral vagina, cervix, and uterus) still contained substantial aPGT121 concentrations, even though only rostral vagina and cervix were directly transfected by aerosol. Taking into account that the GPI membrane anchor is not a permanent tether and proteins can be cleaved by endogenous glycolipidases, it is possible that GPI anchored PGT121 is able to associate with cell membranes of non-transfected cells, as suggested by prior studies. In the case of genital secretions, the source of aPGT121 is likely a combination of enzyme GPI anchor cleavage and cell membranes, whether in the form of non-adhered cells or as fragments, such as exosomes.

The average PGT121 concentrations in both genital secretions and mucosal epithelium remained above the predicted IC50 concentration of 30 ng/mL at 30 days. Ultimately, in vivo, both compartments will contribute to protecting against HIV infection as virus is introduced into the vaginal lumen and diffuses into the mucosa in search of underlying target cells.

Rapid expression kinetics were observed in sheep and rhesus macaque models (less than 4 hours) suggesting that aerosolized mRNA transfection may serve as an acute therapeutic intervention at the site of infection. If cervicovaginally-expressed bNAbs have the potential to follow the same diffusion and trafficking routes as cell-free virions, mRNA transfection may be used in combination with parenteral bNAbs to prevent or attenuate initial virus seeding. PET/CT tracking of mRNA further suggests that mRNA reaches systemic lymph nodes within 70 minutes after aerosol administration; mRNA expression in these secondary lymphoid organs was not evaluated in this study, but future work will explore this observation further. The majority of mRNA remained in the FRT, which is useful in preventing liver expression and toxicity, and localized antibody production may mitigate development of anti-antibody responses.

Another benefit of this platform lies in the relatively simple formulation with water. When lyophilized, mRNA is extremely stable and is not dependent on cold chain storage conditions. The observed robust transfections, simplicity of the aerosolizer, low cost of IVT mRNA, all support the potential for on-demand use as a self-applied prophylaxis technique. Additionally, this platform provides a means to combine multiple therapeutics into a single formulation, as reported here with the simultaneous expression of antibody heavy and light chains, and in our previous study on RSV, overcoming the limits imposed by the use of delivery vehicles. Any theoretical combination of neutralizing or non-neutralizing antibodies that target non-overlapping gp120 and gp41 epitopes could be co-expressed. There is some evidence that chronic controllers do this naturally and develop multiple bNAbs targeting different, non-overlapping sites on Env to suppress virus. Using optimal CD4bs, V3, and MPER targeting antibodies simultaneously could result in IC₅₀'s as low as 0.01 pg/mL, with 100% breadth' Alternatively, bispecific and trispecific ScFv antibodies consisting of the binding domains of multiple bNAbs, increase the breadth of coverage and display high binding affinities, and can be expressed from a single mRNA transcript comparable in size to the ones used in this study. Other sexually transmitted infections that correlate with HIV, such as herpes, hepatitis B, and genital warts (HPV), could be concomitantly targeted with relevant antibodies or anti-microbial peptides.

The disclosed compositions provide a complementary mRNA-based approach to large dose systemic antibody injection to achieve rapid (less than 4 hours) and long-lasting (at least 28 days) neutralizing antibody concentrations within the lower female reproductive tract, providing a firm basis and rationale for an in vivo SHIV challenge in non-human primates, and a possible platform paradigm shift for the prevention and treatment of sexually transmitted infections.

### VI. A. Pharmaceutical Compositions

Pharmaceutical compositions including the disclosed nucleic acid constructs are provided. Pharmaceutical compositions containing the nucleic acid construct can be for administration by parenteral (intramuscular, intraperitoneal, intravenous (IV) or subcutaneous injection), transdermal (either passively or using iontophoresis or electroporation), or transmucosal (nasal, vaginal, rectal, or sublingual) routes of administration or using bioerodible inserts and can be formulated in dosage forms appropriate for each route of administration.

In some in vivo approaches, the compositions disclosed herein are administered to a subject in a therapeutically effective amount. As used herein the term “effective amount” or “therapeutically effective amount” means a dosage sufficient to treat, inhibit, or alleviate one or more symptoms of the disorder being treated or to otherwise provide a desired pharmacologic and/or physiologic effect. The precise dosage will vary according to a variety of factors such as subject-dependent variables (e.g., age, immune system health, etc.), the disease, and the treatment being effected.

For the disclosed nucleic acid constructs, as further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired. For the disclosed nucleic acid constructs, generally dosage levels of 0.001 to 20 mg/kg of body weight daily are administered to mammals. Generally, for intravenous injection or infusion, dosage may be lower.

In certain embodiments, the nucleic acid constructis administered locally, for example by injection directly into a site to be treated. Typically, the injection causes an increased localized concentration of the nucleic acid constructcomposition which is greater than that which can be achieved by systemic administration. The nucleic acid constructcompositions can be combined with a matrix as described above to assist in creating an increased localized concentration of the polypeptide compositions by reducing the passive diffusion of the polypeptides out of the site to be treated.

### 1. Formulations for Parenteral Administration

In some embodiments, compositions disclosed herein, including those containing peptides and polypeptides, are administered in an aqueous solution, by parenteral injection. The formulation may also be in the form of a suspension or emulsion. In general, pharmaceutical compositions are provided including effective amounts of a peptide or polypeptide, and optionally include pharmaceutically acceptable diluents, preservatives, solubilizers, emulsifiers, adjuvants and/or carriers. Such compositions optionally include one or more for the following: diluents, sterile water, buffered saline of various buffer content (e.g., Tris-HCl, acetate, phosphate), pH and ionic strength; and additives such as detergents and solubilizing agents (e.g., TWEEN 20 (polysorbate-20), TWEEN 80 (polysorbate-80)), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

### 2. Formulations for Topical Administration

The disclosed nucleic constructs can be applied topically. Topical administration does not work well for most peptide formulations, although it can be effective especially if applied to the lungs, nasal, oral (sublingual, buccal), vaginal, or rectal mucosa.

Formulations for administration to the mucosa will typically be spray dried drug particles, which may be incorporated into a tablet, gel, capsule, suspension or emulsion. Standard pharmaceutical excipients are available from any formulator.

Transdermal formulations may also be prepared. These will typically be ointments, lotions, sprays, or patches, all of which can be prepared using standard technology. Transdermal formulations may require the inclusion of penetration enhancers.

### 3. Controlled Delivery Polymeric Matrices

The nucleic constructs disclosed herein can also be administered in controlled release formulations. Controlled release polymeric devices can be made for long term release systemically following implantation of a polymeric device (rod, cylinder, film, disk) or injection (microparticles). The matrix can be in the form of microparticles such as microspheres, where the agent is dispersed within a solid polymeric matrix or microcapsules, where the core is of a different material than the polymeric shell, and the peptide is dispersed or suspended in the core, which may be liquid or solid in nature. Unless specifically defined herein, microparticles, microspheres, and microcapsules are used interchangeably. Alternatively, the polymer may be cast as a thin slab or film, ranging from nanometers to four centimeters, a powder produced by grinding or other standard techniques, or even a gel such as a hydrogel.

Either non-biodegradable or biodegradable matrices can be used for delivery of nucleic acids constructs, although in some embodiments biodegradable matrices are preferred. These may be natural or synthetic polymers, although synthetic polymers are preferred in some embodiments due to the better characterization of degradation and release profiles. The polymer is selected based on the period over which release is desired. In some cases linear release may be most useful, although in others a pulse release or “bulk release” may provide more effective results. The polymer may be in the form of a hydrogel (typically in absorbing up to about 90% by weight of water), and can optionally be crosslinked with multivalent ions or polymers.

The matrices can be formed by solvent evaporation, spray drying, solvent extraction and other methods known to those skilled in the art. Bioerodible microspheres can be prepared using any of the methods developed for making microspheres for drug delivery, for example, as described by Mathiowitz and Langer, J. Controlled Release, 5:13-22 (1987); Mathiowitz, et al., Reactive Polymers, 6:275-283 (1987); and Mathiowitz, et al., J. Appl. Polymer Sci., 35:755-774 (1988).

In some embodiments the pharmaceutical composition can include a second therapeutic for treating HIV or other viral infections.

### B. Methods of Use

One embodiment provides a method for prophylactically inhibiting or reducing HIV infection of a female subject comprising the steps of transfecting cervicovaginal epithelial cells in the subject with the construct encoding a PGT121 antibody or antigen binding fragment thereof wherein the antibody or antigen binding fragment is modified to include a GPI membrane anchor. In some embodiments, the construct is an mRNA construct.

Another embodiment provides a method for prophylactically inhibiting or reducing HIV infection in a female subject comprising the step of administering the pharmaceutical composition consisting of the mRNA construct encoding a that encodes an antibody or an antigen binding fragment thereof that specifically binds to an HIV protein, for example gp120, and inhibits or reduces the ability of the HIV virus to infect human cells wherein the antibody is modified to include a GPI membrane anchor and water. In some embodiments the construct is delivered as an aerosol. In other embodiments, the construct is delivered using nanoparticles, for example lipid nanoparticles containing polyethylenimine (PEI) or modified PEI. In some embodiments the construct can be delivered using poly-beta-amino-esters nano-vehicles (PBAEs), and modified PBAEs.

A typical subject is a human, female. An effective amount of a nucleic acid construct encoding an antibody or an antigen-binding fragment thereof is delivered to the reproductive tract of the subject to inhibit, reduce, HIV infection in the subject. The construct transfects cells in the female reproductive tract, for example vaginal and cervical epithelial cells and is expressed. The expressed antibody then binds to HIV proteins for example in viral particles. The antibody-bound virus particles cannot infect cells of the subject

### V. Methods of Manufacture

### A. Methods of Making Antibodies

The disclosed anti-sperm antigen antibodies can be generated in cell culture, in phage, or in various animals, including but not limited to cows, rabbits, goats, mice, rats, hamsters, guinea pigs, sheep, dogs, cats, monkeys, chimpanzees, and apes. Therefore, in one embodiment, an antibody is a mammalian antibody. Phage techniques can be used to isolate an initial antibody or to generate variants with altered specificity or avidity characteristics. Such techniques are routine and well known in the art. In one embodiment, the antibody is produced by recombinant means known in the art. For example, a recombinant antibody can be produced by transfecting a host cell with a vector comprising a DNA sequence encoding the antibody. One or more vectors can be used to transfect the DNA sequence expressing at least one VL and one VH region in the host cell. Exemplary descriptions of recombinant means of antibody generation and production include Delves, Antibody Production: Essential Techniques (Wiley, 1997); Shephard, et al., Monoclonal Antibodies (Oxford University Press, 2000); Goding, Monoclonal Antibodies: Principles And Practice (Academic Press, 1993); Current Protocols In Immunology (John Wiley & Sons, most recent edition).

The disclosed anti-sperm antigen antibodies can be modified by recombinant means to increase greater efficacy of the antibody in mediating the desired function. Thus, it is within the scope of the invention that antibodies can be modified by substitutions using recombinant means. Typically, the substitutions will be conservative substitutions. For example, at least one amino acid in the constant region of the antibody can be replaced with a different residue. See, e.g., U.S. Pat. Nos. 5,624,821, 6,194,551, Application No. WO 9958572; and Angal, et al., Mol. Immunol. 30:105-08 (1993). The modification in amino acids includes deletions, additions, and substitutions of amino acids. In some cases, such changes are made to reduce undesired activities, e.g., complement-dependent cytotoxicity. Frequently, the antibodies are labeled by joining, either covalently or non-covalently, a substance which provides for a detectable signal. A wide variety of labels and conjugation techniques are known and are reported extensively in both the scientific and patent literature. These antibodies can be screened for binding to proteins, polypeptides, or fusion proteins of FLRT3. See, e.g., Antibody Engineering: A Practical Approach (Oxford University Press, 1996).

For example, suitable antibodies with the desired biologic activities can be identified using in vitro assays including but not limited to: proliferation, migration, adhesion, soft agar growth, angiogenesis, cell-cell communication, apoptosis, transport, signal transduction, and in vivo assays such as the inhibition of tumor growth. The antibodies provided herein can also be useful in diagnostic applications. As capture or non-neutralizing antibodies, they can be screened for the ability to bind to the specific antigen without inhibiting the receptor-binding or biological activity of the antigen. As neutralizing antibodies, the antibodies can be useful in competitive binding assays.

Antibodies that can be used in the disclosed compositions and methods include whole immunoglobulin (i.e., an intact antibody) of any class, fragments thereof, and synthetic proteins containing at least the antigen binding variable domain of an antibody. The variable domains differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies.

Also disclosed are fragments of antibodies which have bioactivity. The fragments, whether attached to other sequences or not, include insertions, deletions, substitutions, or other selected modifications of particular regions or specific amino acids residues, provided the activity of the fragment is not significantly altered or impaired compared to the non-modified antibody or antibody fragment.

Techniques can also be adapted for the production of single-chain antibodies specific to an antigenic peptide. Methods for the production of single-chain antibodies are well known to those of skill in the art. A single chain antibody can be created by fusing together the variable domains of the heavy and light chains using a short peptide linker, thereby reconstituting an antigen binding site on a single molecule. Single-chain antibody variable fragments (scFvs) in which the C-terminus of one variable domain is tethered to the N-terminus of the other variable domain via a 15 to 25 amino acid peptide or linker have been developed without significantly disrupting antigen binding or specificity of the binding. The linker is chosen to permit the heavy chain and light chain to bind together in their proper conformational orientation.

Divalent single-chain variable fragments (di-scFvs) can be engineered by linking two scFvs. This can be done by producing a single peptide chain with two VH and two VL regions, yielding tandem scFvs. ScFvs can also be designed with linker peptides that are too short for the two variable regions to fold together (about five amino acids), forcing scFvs to dimerize. This type is known as diabodies. Diabodies have been shown to have dissociation constants up to 40-fold lower than corresponding scFvs, meaning that they have a much higher affinity to their target. Still shorter linkers (one or two amino acids) lead to the formation of trimers (triabodies or tribodies). Tetrabodies have also been produced. They exhibit an even higher affinity to their targets than diabodies.

A monoclonal antibody is obtained from a substantially homogeneous population of antibodies, i.e., the individual antibodies within the population are identical except for possible naturally occurring mutations that may be present in a small subset of the antibody molecules. Monoclonal antibodies include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

Monoclonal antibodies can be made using any procedure which produces monoclonal antibodies. In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

Antibodies may also be made by recombinant DNA methods. DNA encoding the disclosed antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques.

Methods of making antibodies using protein chemistry are also known in the art. One method of producing proteins comprising the antibodies is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, CA). One skilled in the art can readily appreciate that a peptide or polypeptide corresponding to the antibody, for example, can be synthesized by standard chemical reactions. For example, a peptide or polypeptide can be synthesized and not cleaved from its synthesis resin whereas the other fragment of an antibody can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. Alternatively, the peptide or polypeptide is independently synthesized in vivo as described above. Once isolated, these independent peptides or polypeptides may be linked to form an antibody or antigen binding fragment thereof via similar peptide condensation reactions.

For example, enzymatic ligation of cloned or synthetic peptide segments allow relatively short peptide fragments to be joined to produce larger peptide fragments, polypeptides or whole protein domains. Alternatively, native chemical ligation of synthetic peptides can be utilized to synthetically construct large peptides or polypeptides from shorter peptide fragments. This method consists of a two-step chemical reaction. The first step is the chemoselective reaction of an unprotected synthetic peptide-alpha-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site.

### B. Methods for Producing Isolated Nucleic Acid Molecules

Isolated nucleic acid molecules can be produced by standard techniques, including, without limitation, common molecular cloning and chemical nucleic acid synthesis techniques. For example, polymerase chain reaction (PCR) techniques can be used to obtain an isolated nucleic acid encoding a variant polypeptide. PCR is a technique in which target nucleic acids are enzymatically amplified. Typically, sequence information from the ends of the region of interest or beyond can be employed to design oligonucleotide primers that are identical in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. General PCR techniques are described, for example in PCR Primer: A Laboratory Manual, ed. by Dieffenbach and Dveksler, Cold Spring Harbor Laboratory Press, 1995. When using RNA as a source of template, reverse transcriptase can be used to synthesize a complementary DNA (cDNA) strand. Ligase chain reaction, strand displacement amplification, self-sustained sequence replication or nucleic acid sequence-based amplification also can be used to obtain isolated nucleic acids. See, for example, Lewis (1992) Genetic Engineering News 12:1; Guatelli et al. (1990) Proc. Natl. Acad. Sci. USA 87:1874-1878; and Weiss (1991) Science 254:1292-1293.

Isolated nucleic acids can be chemically synthesized, either as a single nucleic acid molecule or as a series of oligonucleotides (e.g., using phosphoramidite technology for automated DNA synthesis in the 3′ to 5′ direction). For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase can be used to extend the oligonucleotides, resulting in a single, double-stranded nucleic acid molecule per oligonucleotide pair, which then can be ligated into a vector. Isolated nucleic acids can also obtained by mutagenesis. Protein-encoding nucleic acids can be mutated using standard techniques, including oligonucleotide-directed mutagenesis and/or site-directed mutagenesis through PCR. See, Short Protocols in Molecular Biology. Chapter 8, Green Publishing Associates and John Wiley & Sons, edited by Ausubel et al, 1992.

### EXAMPLES

### Materials and Methods

### Cell Lines and Virus Culture

Vero and HEK 293 cells were obtained from ATCC and cultured in RPMI1640 media supplemented with L glutamine, 10% fetal bovine serum, and antibiotics.

SHIV162p3 (simian immunodeficiency virus SIVmac239 backbone with an HIV-1 Glade B, R5-tropic envelope) was provided by Dr. Nancy Miller, National Institute of Allergy and Infectious Diseases, National Institutes of Health. SHIV2873Nip (SIVmac239 backbone with an HIV-1 Glade C, R5-tropic envelope isolated from a Zambian infant) was obtained from Dr. Ruth Ruprecht, the Texas Biomed AIDS Research Program, Texas Biomedical Research Institute. Both viruses were propagated in rhesus peripheral blood mononuclear cells activated with Concanavalin A and recombinant IL-2. Virus stocks were titrated in TZM-bl cells to derive the 50% tissue culture infective dose (TCID₅₀). For neutralization assay, both viruses were used at a concentration that resulted in 10 relative 10⁵ relative light units (RLU) with a repeat titration of viral stocks in parallel to each assay.

### Particle Mediated In Vitro Transfections

Vero or HEK293 cells were transfected using either lipofectamine 2000 (Thermo Scientific) or Neon electroporation system (Invitrogen), according to the manufacturer's instructions, into a 24 well plate (for imaging) and were transfected with the indicated amount of mRNA per 200,000 cells. For mRNA encoding whole IgG, heavy chain and light chain mRNAs were combined in a 4:1 mass ratio for equimolar conditions. Twenty hours post-transfection, cells were fixed and immunostained with or without permeabilization.

### Immunostaining

Vero or HEK293 cells were fixed with 4% paraformaldehyde (PFA) (Electron Microscopy Sciences) for 10 min at room temperature before permeabilization with 0.2% Triton X-100 (Sigma) for 5 min at room temperature. Then, cells were blocked by incubation with 5% bovine serum albumin (Calbiochem) for 30 min at 37° C. before being incubated with primary antibody for 30 min at 37° C. Cells were then washed with PBS and incubated with secondary antibody for 30 min at 37° C. Multiple antibody labeling was performed simultaneously after checking cross-reactivity. Nuclei were then stained with 4′,6-diamidino-2-phenylindole (DAPI) (Life Technologies), and coverslips were mounted onto glass slides with Prolong Gold (Life Technologies).

Synthetic mRNA In Vitro Transcription

The anchored and secreted PGT121 sequences were ordered as a DNA gBlock from IDT (sequences found in Table 1) containing a 5′ UTR with Kozak sequence, a 3′ UTR derived from the mouse alpha globin sequence, and extensions to allow for Gibson assembly. The sequences were human codon optimized using the IDT website. The gBlock was cloned into a PCR amplified pMA7 vector (primers B1 and B2, Table 2) through Gibson assembly using NEB Builder with 3× molar excess of insert. All reaction transcripts were 0.8% agarose gel purified prior to assembly reaction. Subsequent plasmids from each colony were Sanger sequenced to ensure desired sequence fidelity.

Plasmids were linearized with NotI-HF (New England BioLabs) overnight at 37° C. Linearized templates were purified by sodium acetate (Thermo Scientific) precipitation before being rehydrated with nuclease free water. IVT was performed overnight at 37° C. using the HiScribe T7 kit (NEB) following the manufacturer's instructions. RNA product was treated with DNase I (Aldevron) for 30 minutes to remove template and purified using lithium chloride precipitation (Thermo Scientific). RNA was heat denatured at 65° C. for 10 minutes before being capped with a Cap-1 structure using guanylyl transferase (Aldevron) and 2′-O-methyltransferase (Aldevron). Transcripts were then polyadenylated enzymatically (Aldevron). mRNA was then purified by lithium chloride precipitation, treated with alkaline phosphatase (NEB), and purified again. Concentrations were measured using a Nanodrop. mRNA stock concentrations were 3-5 mg/mL. Purified RNA products were analyzed by gel electrophoresis to ensure purity.

TABLE 1
PGT121 concentrations by animal and
mean in sheep secretions (μg/mL)

| | aPGT121 | | | | sPGT121 | | |
|---|---|---|---|---|---|---|---|
| | Animal ID | | | | Animal ID | | |
| Day | 420 | 456 | 461 | Mean | 401 | 414 | Mean |
| 1 | 594.0 | 122.0 | 299.0 | 338.3 | 22.1 | 100.7 | 61.4 |
| 7 | 60.5 | 51.6 | 80.1 | 64.1 | 26.1 | 46.1 | 36.1 |
| 14 | 91.5 | 68.1 | 97.7 | 85.7 | 13.8 | 12.8 | 13.3 |
| 21 | 52.2 | 70.8 | 149.0 | 90.6 | 11.1 | 8.4 | 9.8 |
| 28 | 35.6 | 55.2 | 41.1 | 44.0 | | | |

TABLE 2
Primer sequences

| Primer | | | |
|---|---|---|---|
| Name | Purpose | Sequence | SEQ ID |
| A1: FWD | PCR | GCGACGATTGGCGGAAGGCCGTCAAGGCCGCATTTTTAAG | 3 |
| A2: RVS | Gibson | CGGGCAGTGAGCGGAAGGCCCATGAGGCCCAGTTTTTG | 4 |
| | Overlaps | | |
| | onto | | |
| B1: FWD | Create | GGCCTTCCGCTCACTGCC | 5 |
| B2: RVS | linear | GGCCTTCCGCCAATCGTC | 6 |
| | pMA-7 | | |
| | vector | | |
| | template | | |
| C1: FWD | Sanger | TAAAACGACGGCCAGTGAGCGCGAC | 7 |
| C2: RVS | sequencing | CTGGCACGACAGGTTTCCCGACTGG | 8 |
| | of | | |
| | insert | | |
| | fidelity | | |

### Animals

Yearling nonpregnant female Katandin ewes were sourced from a farm in Starkville Mississippi and housed in the College of Veterinary Medicine Teaching and Research Pastures 11, 12, and 3 at Mississippi State University. All experiments were reviewed and approved by the Mississippi State University IACUC.

Female rhesus macaques (n=3) used in this study were housed in the BSL2+ housing of the New Iberia Research Center and maintained in accordance with the regulations of the Guide for the Care and Use of Laboratory Animal and the studies were reviewed and approved by the University of Louisiana IACUC. The macaques were fed monkey chow (Purina) supplemented daily with fresh fruit or vegetables with water provided ad libitum. All procedures in macaques were conducted under sedation with Telazol and/or ketamine.

### Sheep Aerosol Delivery

Sheep were sedated using 0.2 mg/kg of xylazine administered intravenously. After 3-5 minutes, animals were positioned dorsal recumbency on a flat table. A polyethylene vaginal speculum, with an internal diameter of 15 mm, was coated with sterile lubricant and positioned such that the cervical os was visible. Mucus was cleared away by brief surface cleaning with a cotton tipped applicator. The MADgic, with dosing syringe, was then inserted into the speculum until the distal nozzle of the aerosolizer was 5 mm from the distal opening of the speculum. Hand pressure was then used to spray the mRNA. If the vagina was also being sprayed in the same animal, the speculum was removed in a caudal direction by 2-4 cm, surface cleaning was performed, and the vagina was sprayed with a freshly loaded dose of mRNA. Average volumes delivered ranged from 300-450 pL, depending on the experiment. Upon completion of the procedure the sheep was placed in sternal recumbency and sedation was reversed via intramuscular administration with 1.4 mg/kg tolazoline when applicable. Speculums were disinfected in 4% chlorhexidine solution and rinsed with distilled water between animals, to minimize potential cross contamination. All medication doses, mRNA doses, times of administration, and other experimental notes were recorded, as required by IACUC at Mississippi State University.

### IVIS Imaging and Image Analysis

Sheep were euthanized by intravenous barbiturate injection and the female reproductive tract was removed and excised. The tract was cut along a single edge so the entire surface area of the vagina and cervix lay flat. Two mL of a fresh 1:40 solution of NanoGlo Substrate in 1×PBS was added to the entire vagina and uterus. After 1 minute at room temperature, the organ was imaged with an IVIS (IVIS lumina XRMS, Series III; supported through USDA-ARS Biophotonics Initiative #58-6402-3-018). The positive regions were identified from the images, and carefully excised. The tissues were then either weighed and snap frozen in liquid nitrogen for protein extraction or placed in 4% paraformaldehyde overnight at 4° C. for microscopic analysis.

Acquired luminescence FRT images were analyzed using Living Image Software (PerkinElmer). Relevant regions of interest (ROIs) were identified from background tissue by high pass thresholding at 10%. The average radiance for each ROI was measured and control average radiance from a negative control sample was used for background subtraction purposes.

### Antibodies

For immunostaining, the primary antibodies used were mouse anti-CD63 (Developmental Studies Hybridoma Bank, Cat. No. H5C6), mouse anti-clathrin light chain (Biolegend, Cat. No. MMS-423P), mouse anti-caveolin (Abcam, Cat. No. ab17052), LAMP1 (Developmental Studies Hybridoma Bank, Cat. No. H4A3), mouse anti-EEA1 (BD Biosciences, Cat. No. 610456), and rabbit anti-NanoLuc® (Promega). All primary antibodies for immunostaining experiments were used at 1 μg/mL. Secondary antibodies used were donkey anti-mouse AlexaFluor 546 (Life Technologies) and donkey anti-rabbit Alexa Fluor 488 (Life Technologies). All secondary antibodies for immunostaining experiments were used at 8 μg/mL.

For western blot, primary antibodies used were rabbit anti-NanoLuc® (Promega), and mouse anti-GAPDH (GeneTex, Cat. No. GT239), diluted to 0.74 pg/mL and 1 pg/mL, respectively, in Odyssey blocking buffer (LI-COR) with 0.1% Tween-20. The secondary antibodies were a donkey anti-mouse IRDye 680RD (LI-COR) and a donkey anti-rabbit IRDye 800 (LI-COR) and were diluted 1:3000 in Odyssey blocking buffer with 0.1% Tween-20.

### Sheep Secretion Collection

Sponges (Beaver-Visitec International Inc.) were presoaked with 50 pL of 1×PBS. If multiple sites within the lower FRT were sampled simultaneously, the sponges were connected using monofilament suture. With the animals standing and non-sedated, vaginal secretions were collected by positioning the sponge in the desired location using forceps and waiting for 3 minutes. The sponges were then removed and placed into 1.5 mL tubes and frozen at −80° C. until analysis. To extract secretions from the sponges, the sponges were first placed into pre-weighed QiaShredder filters and centrifuged at 20,000×g for 10 minutes at 4° C. 200 pL of extraction solution (IGEPAL, protease inhibitor in PBS) was added to the sponge and placed on ice for 15 minutes. The sponge was then spun at 20,000×g for a further 30 minutes. Secretions were aliquoted and stored at −80° C. for further analysis.

### Sheep Tissue Lysate Preparation

Frozen sheep FRT tissues were crushed using a BioPulverizer (BioSpec Products). Radioimmunoassay (RIPA) buffer (Thermo Scientific) was then added at a ratio of 2 μL to 1 mg of tissue. This solution was then further homogenized using a bead mill (Next Advance) before stored frozen at −80° C. Lysate protein concentrations were determined using a bicinchoninic acid assay (BCA, Pierce).

### Macaque Aerosol Delivery

Female rhesus macaques (n=3) were used in this study. Rhesus macaques were sedated with Telazol/ketamine and placed in ventral recumbency. PGT121 mRNA solution, in molecular grade nuclease-free H2O, was loaded into a 1 mL high-pressure syringe (Medline). The syringe was attached to the MADgic Laryngo-Tracheal Mucosal Atomization Device (Teleflex). A modified 3 mL syringe was used as a speculum to visualize the cervix. Mucus from the cervico-vaginal lumen was cleared away by brief surface cleaning with a cotton-tipped applicator. The MADgic, with dosing syringe, was then inserted into the tube until the distal nozzle of the aerosolizer was 5 mm from the distal opening of the speculum. Hand pressure was then used to spray the mRNA solution. If the vagina was also being sprayed in the same macaque, the speculum was removed in a caudal direction by 2-4 cm, and the vagina was sprayed with a freshly loaded dose of mRNA. Each 250 μg dose was 300 μL, while the 400 and 1000 μg dose was 150 μL each to minimize leakage of excess solution.

mRNA Radiolabeling and Distribution by PET/CT Imaging

To study the bio-distribution of IVT mRNA via whole-body PET-CT, radionuclide-labeled antisense oligonucleotides were annealed to the mRNA before delivery, as previously descri 31,32,38. Briefly, two 2′0-Methyl RNA/DNA chimeric oligos (Biosearch Technologi complementary to the mRNA 3′ UTR were purchased with the following sequences: MT1 5′-Thiol-XTTTTTXGCAAGCCCCGCAGAAGX-3′ (SEQ ID NO:9) and MT2 5′-Thiol- TXTTATTXAGAGAAGAAGGGCAXGG-3′ (SEQ ID NO:10) where the boldface indicates 2′-O-Methyl RNA and X indicates T(C6-Amino) modifications.

For chelator conjugation, the 5′ disulfide was reduced by incubation with tris(2-carboxyethyl)phosphine(TCEP) (5 mM) (Thermo Fisher Scientific). Oligonucleotides were then repeatedly diluted in 0.1 M chelexed phosphate buffer pH 7.3 and filtered (3 kDa MWCO, Millipore) to remove the reducing agent. Oligonucleotides were then modified by incubation with DOTA-maleimide and DOTA-NHS ester (10× and 50× molar excess respectively, Macrocyclics) for 6 h at room temperature under gentle agitation.

Unbound chelators were removed by centrifugal filtration (3 kDa MWCO) in 0.1 M chelexed-phosphate buffer, and the individual oligos were quantified by Nanodrop, aliquoted and lyophilized. Oligos were then resuspended in 0.1 M chelexed ammonium acetate pH 5.5 and incubated with ⁶⁴Cu for 1 h at 37° C. Unbound ⁶⁴Cu was removed by centrifugal filtration (3kDA MWCO) in 0.1 M chelexed-phosphate buffer.

To pre-label IVT mRNA before delivery, mRNA was annealed to 0.7× molar excess probes in a thermal cycler with the following optimized protocol: 80° C. for 2 min, gradients from 80° C. to 25° C. in 30 sec steps (1 degree per step), 25° C. for 2 min. The mRNA was immediately resuspended in saline and delivered.

The aerosolized dose of radiolabeled mRNA delivered to rhesus macaques was 0.2 mCi per macaque, delivered in two sequential 300 μL doses using the Teleflex atomizer. PET/CT scanning was undertaken at 70 minutes, 4 hours, 24 hours, and 48 hours post administration using a Philips Gemini TF64 clinical PET/CT scanner.

All quantitative software analysis was completed from DICOM formatted images using MIM Software Inc. (Cleveland, Ohio). A high pass thresholding filter set at 28% (i.e. only the highest 72% of signal within a given region) was used to assign region of interests (ROIs) for each lymph node. These volumetric regions of interest were used to report the total standard uptake values (SUV) values. To account for instrument parameters that might have altered between imaging sessions, contralateral muscle SUV average values for a circumscribed ROI (region of interest) were used to normalize readings.

### SHIV Neutralization Titers

The neutralization activity of antibody in macaque vaginal secretions against either SHIV162p3 (clade B) or SHIV2873Nip (clade C) was measured using the reference protocol of the luciferase-based HIV-1 neutralization assay in TZM-bl cells (Dr. Montefiori laboratory, Duke University). Briefly, 50 μl of 5-fold serial dilutions of vaginal secretions and 50 μl of titrated virus (105 RLU) were incubated for 1 hour at 37° C. in a 96-well flat-bottom plate. Next, 100 μl of TZM-bl cells (1×104/well) in 10% DMEM growth medium containing 30 μg/ml DEAE dextran (Sigma-Aldrich) were added to each well, and the 96-well plates were incubated for 48 hours. Assay controls included TZM-bl cells alone (cell control, no virus) and TZM-bl cells with virus only (virus control, no test reagent). At 48 hours, the cells were lysed and luciferase activity was measured using a BioTek Synergy HT multimode microplate reader. The average background luminescence (RLU) from cell control wells was subtracted from the luminescence for each experimental well, and infectivity curves were generated using GraphPad Prism (v7.01) software, where values from the experimental wells were compared against the value from virus control wells. The 50% inhibitory concentration (IC50) was calculated based on the vaginal secretions dilution that caused a 50% reduction of RLU compared to the virus control wells after subtraction of cell control RLU.

To measure the neutralization activity of mRNA-expressed antibodies, Vero cells were seeded into 75 cm2 culture flasks and transfected with either aPGT121 or sPGT121 HC mRNA and NanoLuc® or non-NanoLuc® PGT121 LC mRNA. For aPGT121 constructs, the cells were lysed using RIPA buffer and clarified by centrifugation for 20 minutes at 18,000×g at 4° C. The supernatant was then collected and stored at −80° C. until further use. For sPGT121 constructs, the culture media supernatant was concentrated using a 10 kDa MWCO centrifugal filter before being stored at −80° C. until further use. The cell lysates (for aPGT121) and cell supernatants (for sPGT121) were purified using a NAb Protein A Plus spin column (Pierce) before being assayed for neutralization activity as described above. mRNA-expressed antibodies were compared to PGT121-N (a gift from Mapp Biopharmaceutical Inc.). Neutralization activity from all constructs were compared using molarity to account for molecular weight difference due to the inclusion of the membrane anchor or NanoLuc® reporter.

### SHIV ADCC Assay

ADCC was assessed as previously described51. Briefly, CEM.NKR.CCR5.CD4+-Luc target cells were infected with 50 ng SHIV162p3 or SHIV2873Nip by spinoculation and cultured for 4 days. Two-fold serial dilutions of each PGT antibody, either mRNA-expressed (aPGT121-NLuc, aPGT121, sPGT121-NLuc, or sPGT121) were added to the infected targets for 20 min at room temperature. An NK cell line CD16-KHYG-1, as effector cells, were added at a 10:1 effector to target ratio and these were incubated for additional 8 hours. The cells were then lysed and luciferase activity (RLU) was measured using a luminometer (Synergy HT, Bio-Tek).

### Macaque Explant SHIV Challenge

At 24 post transfection, biopsies from the indicated regions of the FRT in transfected macaques were collected. Tissues were then washed 3× with 1×PBS, incubated with 5.5×104 TCID50 of SHIV162p3 for 2 hours at 37° C., washed a further 3×, and then cultured on collagen sponges in 10% FBS supplemented DMEM media. Supernatant from the cultures was collected at the indicated time points and frozen, until p27 ELISA results could be attained by ELISA.

NanoLuc® Luciferase gp120 ELISA

The gp120 ELISA protocol was adapted from Burton, 200118. Recombinant gp120JR-FL (MyBioSource) was coated to the wells of a microtiter plate (Corning) at a concentration of 2 pg/ml by incubation overnight at 4° C. in 1×PBS. The plates were washed four times with PBS-0.05% Tween-20 and blocked with 3% bovine serum albumin. Following washing, vaginal secretions or purified PGT121-NanoLuc® stocks were applied to the plate and incubated for 2 hours at 37° C. A PGT121-NanoLuc® antibody standard curve was run on each plate, as necessary. After washing, a 1:50 ratio of NanoGlo in 1'PBS was added to each well. After 1 minute, a BioTek Synergy H4 Microplate reader set at an emission spectrum of 450 nm and 2s integration time was used to record the luminescence in each sample.

### Western Blot

The indicated quantity of tissue lysates or secretions were mixed with 4x SDS loading buffer (LI-COR Biosciences), boiled for 10 min at 70° C., chilled on ice, and loaded into wells of a Bolt 4-12% Bis-Tris Bolt precast gel (Life Technologies) alongside a molecular weight marker (LI-COR). Gel was run in a Mini Gel Tank system (Life Technologies) in 1X MOPS running buffer (Life Technologies) at a constant 200V for 32 min. Protein was then transferred to 0.45 μm pore nitrocellulose membranes (Life Technologies) in 1× Bolt western transfer buffer (Life Technologies) at a constant 12 V for 1 h using a Mini gel blot module (Life Technologies).

Nonspecific binding to blot was blocked using PBS Odyssey blocking buffer (LI-COR) at room temperature. Primary antibody was then applied in blocking solution with 0.1% Tween-20 (VWR) and allowed to incubate overnight at 4° C. Blots were washed three times with 1×PBS containing 0.1% Tween-20 (PBST). Secondary antibody was then applied and allowed to incubate for 1 hr before blots were again washed three times with PBST. Blots were imaged using an Odyssey CLx IR scanner (LI-COR). Only linear contrast enhancements were performed for the final representative images.

For quantification, a standard curve dilution series of purified PGT121 was loaded in the same gel as the samples in all cases. A linear regression with interpolation was then performed on the densitometry of the detected band and samples.

Statistical analysis

Results were plotted, and statistical analyses were performed using Prism 8 (GraphPad, La Jolla, California). Power analysis was performed to ensure adequate sample size for experiments. Hypothesis tests were chosen and performed as appropriate, indicated in the figure captions.

### Example I: Aerosolized Transfection Delivers mRNA Into the Cell Cytoplasm

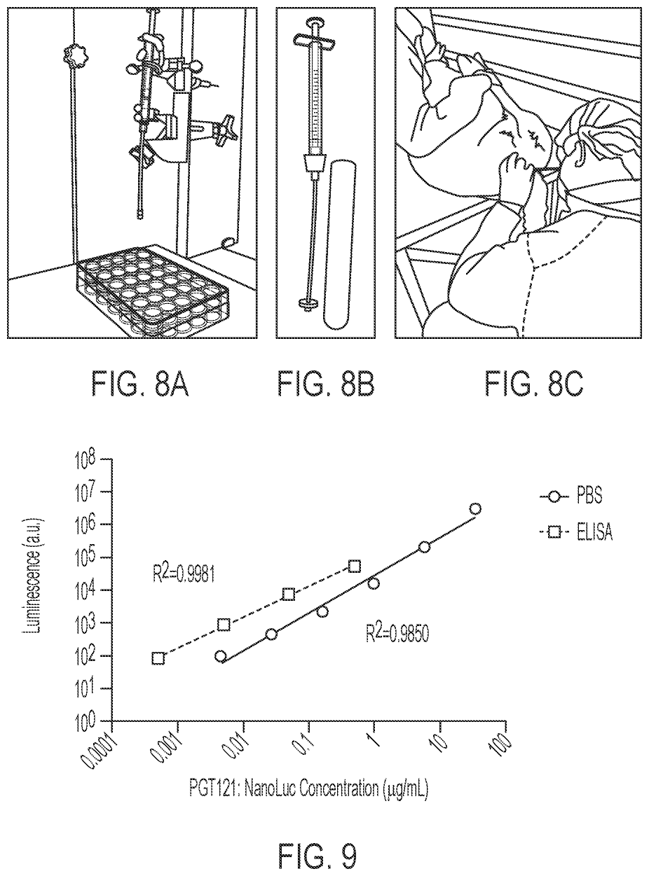

It was hypothesized that aerosolizing mRNA diluted in water may transfect other mucosal interfaces, such as the FRT. This method avoids induction of innate immunity and inflammation, which can be activated by many common synthetic mRNA carriers and facilitates potential translation to the clinic. The use of water as an mRNA solvent is substantiated by prior research, which demonstrated that hypotonic formulations markedly increased the rate at which small molecule drugs and muco-inert nanoparticles reached the vaginal epithelial surface in mice or rectal epithelial tissues in monkeys. A vertical in vitro apparatus to transfect cell monolayers with aerosolizers (FIGS. 8A and 8B).

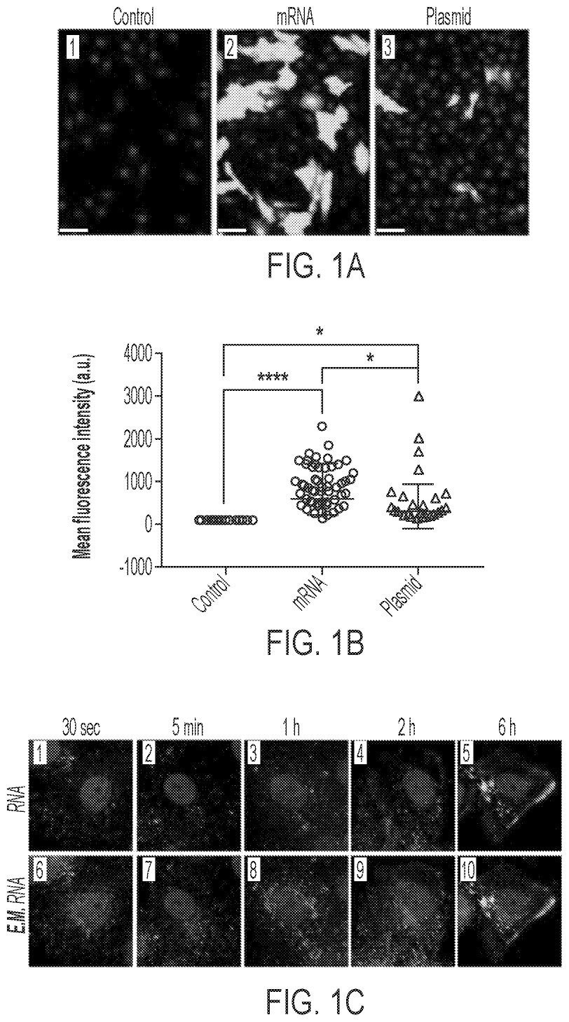

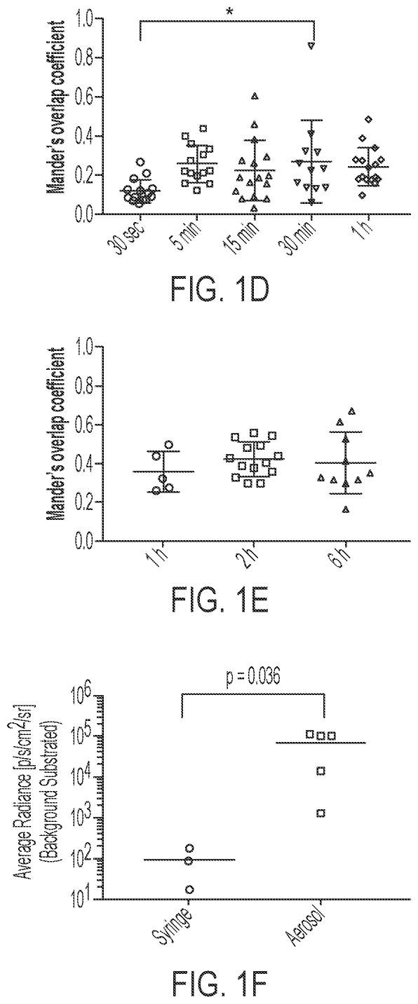

Upon aerosol transfection to Vero cells, the mean fluorescence intensity (MFI) of mRNA-encoded GFP was significantly higher compared to either the pDNA-encoded or control treatments (FIGS. 1A and 1B).

To initially investigate the uptake mechanism involved with aerosol-based transfection, monolayers of adherent cells were transfected with Dylight™ 650 fluorescently labeled GFP mRNA, through the use of complementary oligos annealed to its 3′untranslated region (UTR) 31-33. At discrete time points after spraying, the fraction of nucleic acid outside of the endosomal compartment or nucleus was estimated using fluorescence microscopy (FIGS. 1C1-1C10). Approximately 70% of aerosolized GFP mRNA was found to be cytosolic 15 minutes post-delivery (FIG. 1D). There was a slight, but not significant, increase in endosome localization over time (up to 6 hours) (FIG. 1E). Therefore, aerosol transfection likely delivers unformulated nucleic acids directly into the cytosol of cells, allowing rapid access to the ribosomal machinery and subsequent protein expression.

### Example II mRNA Aerosolization is Required to Transfect the Cervix Epithelium

The spatio-temporal parameters governing FRT transfection in sheep, which have geometrically similar FRT compared to humans were explored. In the sheep used in this study, the distance from the vaginal vestibule to the cervix was 9-11 inches. 250 pg of Firefly Luciferase encoding mRNA in water was delivered to the FRT via a MADgic Teleflex aerosolizer (FIGS. 8B-8C). After 24 hours, the entire FRT was removed during postmortem necropsy, D-Luciferin substrate was applied, and an In Vivo Imaging System (IVIS) was used to record the bioluminescence signal. As an alternative delivery method, mRNA was “squirted” by high-pressure syringe onto the cervix.

Robust transfection of the cervix was observed by aerosolization, but no signal was detected using a high-pressure syringe (FIG. 1F). These data indicate that aerosolization is a reliable means to transfect the cervix epithelium with synthetic mRNA in water.

### Example III: PGT121-NanoLuc® Anchored to the Membrane of Cells Binds SHIV

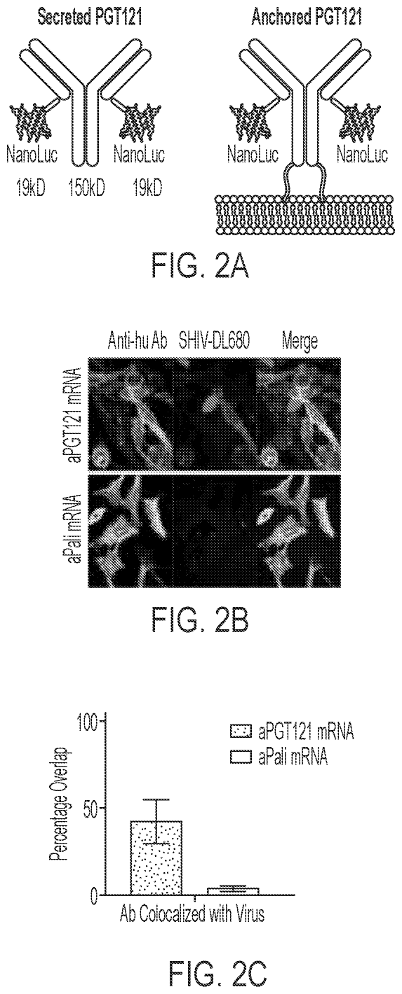

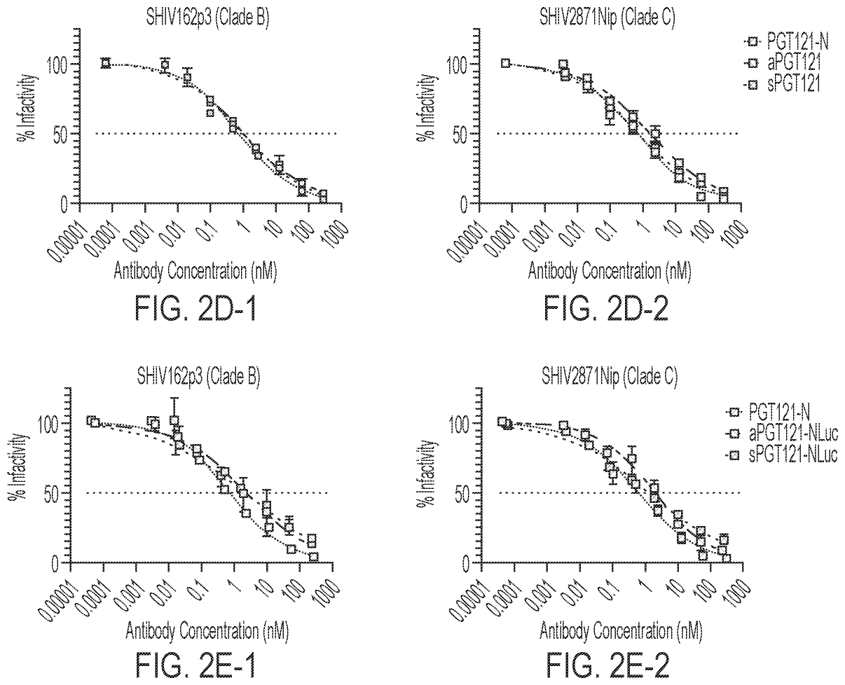

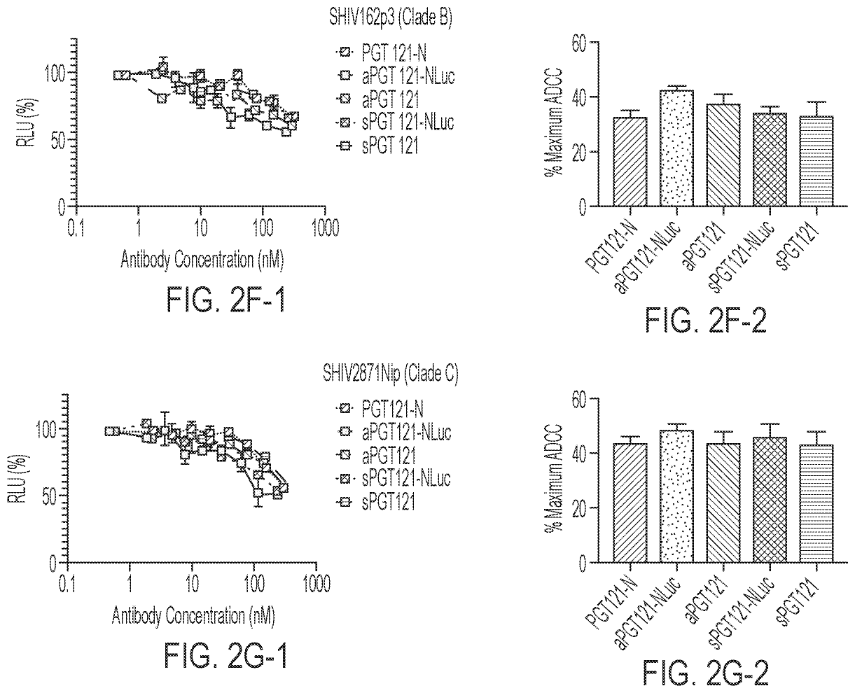

To ensure that experimentally relevant epithelium tissue was examined during necropsy and subsequent tissue processing, a luminescence reporter, nanoluciferase (NanoLuc®), was fused to the 3′ end of PGT121 light chain (LC) (FIG. 2A). Secreted PGT121-NanoLuc (sPGT121-NanoLuc®) was isolated by gentle protein A/G purification of the supernatant after transient transfection of adherent cells. The dynamic range for PGT121-NanoLuc® in solution was found to be nearly six orders of magnitude (FIG. 9). In a modified gp120 ELISA assay a sensitivity of approximately 500 pg/mL was observed, on par with conventional ELISA readouts, but with a greater dynamic range (FIG. 9).

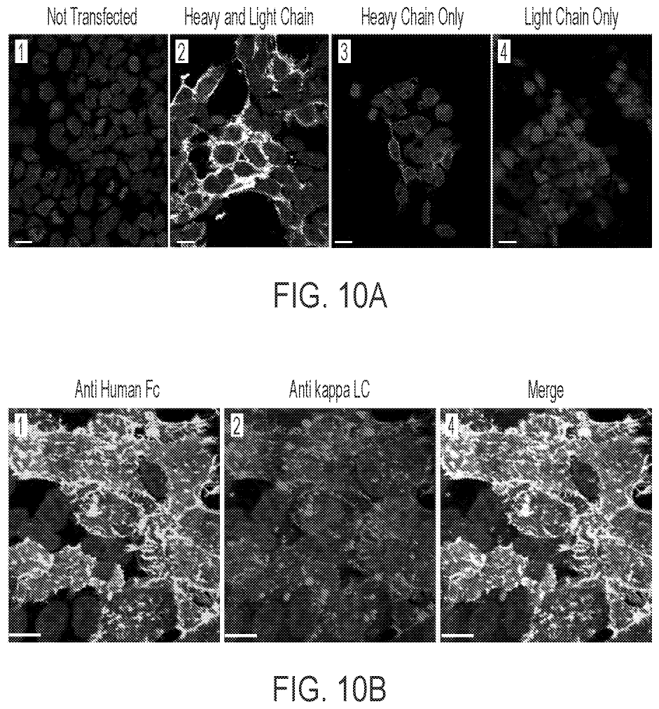

Additionally, the GPI anchor of decay associated factor (DAF) was fused to the 3′ end of the heavy chain (HC) domain of the PGT121 mRNA transcript (FIG. 2A). The complete IgG, anchored PGT121 (aPGT121), efficiently localized to the cell surface when GPI anchored-HC and LC mRNA transcripts were delivered simultaneously at a 1:4 mass ratio (FIG. 10). As expected, the delivery of mRNA encoding either single chain alone did not result in significant staining with an anti-human secondary antibody, indicating incomplete antibody formation.

Expressed aPGT121 retained the ability to bind SHIV virions when displayed on the cell membrane. HEK293 cells were transiently transfected with either aPGT121 or aPali mRNA 24 hours prior to addition of fluorescently labeled SHIV AD8EO virions, and the cells were analyzed by confocal microscopy (FIG. 2B). Colocalization analysis indicated that aPGT121 expressing cells, but not aPali, were able to bind SHIV virions and capture them at the surface of transfected cells (FIG. 2C).

mRNA-expressed aPGT121 and sPGT121 with and without a NanoLuc® was then purified and compared the Glade B (SHIV162p3) and C (SHIV2871Nip) SHIV neutralizing capacity of each construct to a parental PGT121 produced in Nicotiana (PGT121-N). While both the anchored and secreted forms of mRNA-expressed PGT121 without a NanoLuc® were not significantly different from PGT121-N (FIG. 2D), the fusion of the NanoLuc® reporter had a minor effect on the neutralization of both clades (FIG. 2E). Hence, aPGT121-NanoLuc® anchored to the plasma membrane retained Env binding capacity and neutralizing capacity. Finally, to verify that mRNA-expressed antibodies retained similar Fc function to the parental PGT121, we conducted an antibody-dependent cell-mediated cytotoxicity (ADCC) assay using the same Glade B and Glade C SHIV strains, respectively. No detectable difference was measured between any of the mRNA-expressed antibody forms and PGT121-N with either viral Glade (FIGS. 2F-2G). Therefore, mRNA-expressed antibodies, regardless of membrane-anchor or NanoLuc® modification, retain neutralizing capacity and effector cell function comparable to the commercial PGT121.

### Example IV: The Entirety of the Lower FRT can be Transfected by mRNA Aerosol

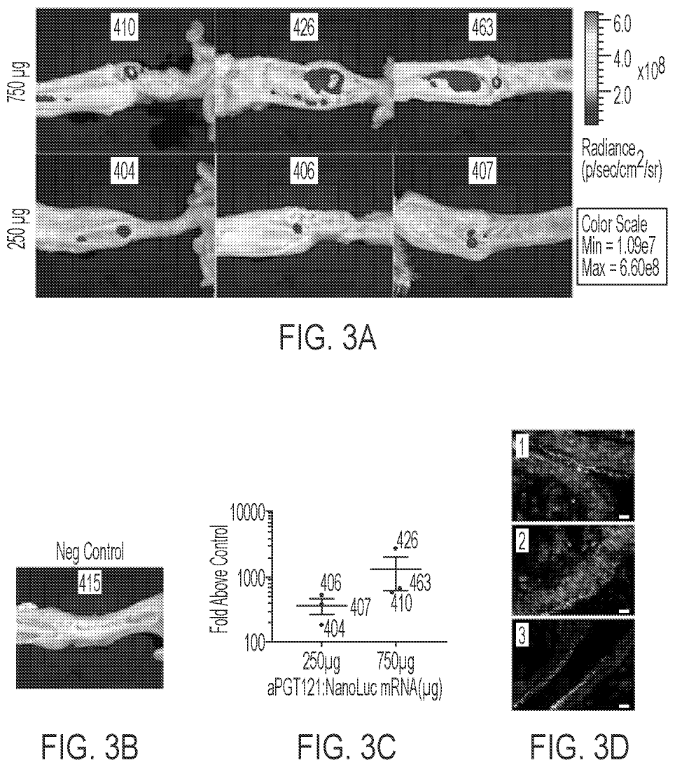

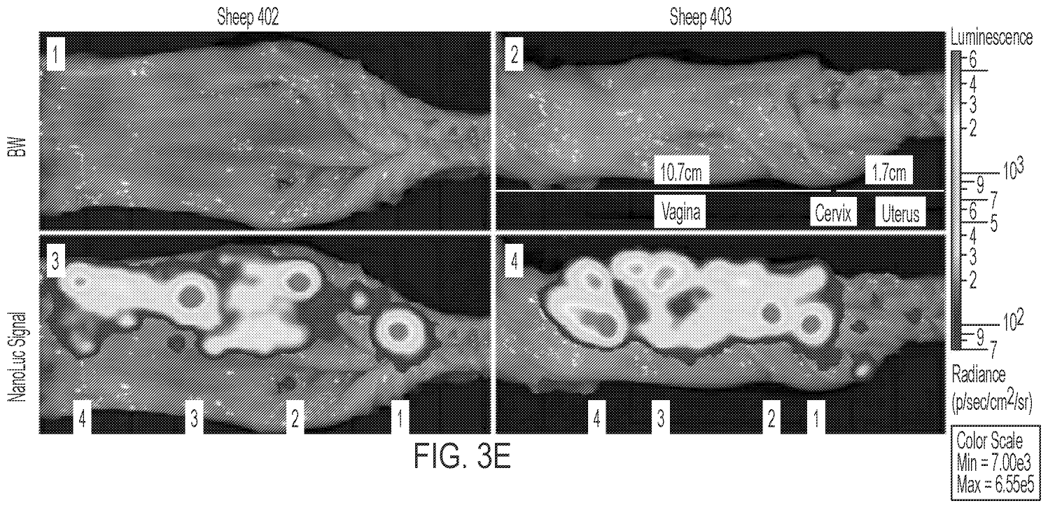

In the luciferase expression experiments in FIG. 1, a dose of 250 pg mRNA was arbitrarily chosen. To determine if higher doses of mRNA lead to production of more antibody, we treated three sheep each with either 250 pg or 750 pg of mRNA; all treatments were administered in a 300 pL volume. After 24 h, the FRT was removed after euthanasia and necropsy and 2 mL of NanoLuc® substrate was applied to cover the entire vaginal, cervical, and uterine epithelium; IVIS imaging was used to observe the location and quantify the luminescence from each animal (FIG. 3A-3E4). Luminescence at and surrounding the cervix was higher in all 750 pg dosed animals (FIGS. 3A-3C). Given the increase in expression, subsequent experiments utilized 750 pg of mRNA per dose.

To visualize mucosal transfection via microscopy, fluorescently labeled mRNA was delivered to the cervix via aerosol. At 24 hrs, FRT tissue was removed, fixed, and immunostained for NanoLuc®, allowing for both mRNA and protein visualization. Expressed aPGT121 was localized to both the cervical epithelial layer and stromal cells (FIGS. 3D1-3D3). Surprisingly, both mRNA and protein expression were detected well below the outer-most epithelial layers. A no primary antibody control demonstrated the specificity of the secondary antibody. Whether both the vaginal and cervical epithelia were equally permissive to transfection was explored. Four sprays, 750 pg each, were applied sequentially starting at the cervix, with 23 cm retractions after each administration (FIGS. 3E1-3E4). The amount and distribution of expressed protein were assessed via IVIS imaging at 24 h. In both tested animals, aPGT121-NanoLuc® expression was detected throughout the vagina and cervix with similar luminescence intensity maximums (FIGS. 3E1-3E4). These data suggest that both the vagina and cervix epithelia can be efficiently transfected via aerosol.

### Example V: Membrane Anchoring Retains PGT121 in Genital Secretions and FRT Epithelium

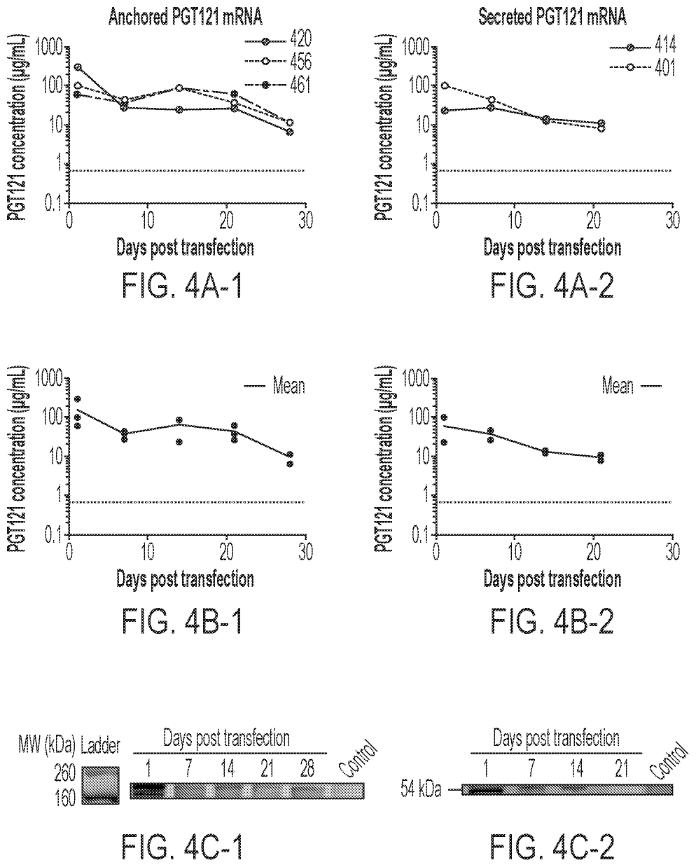

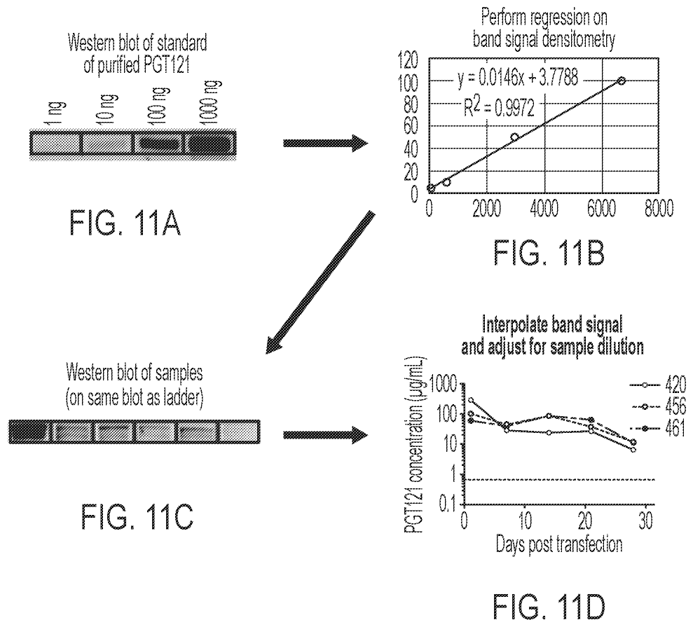

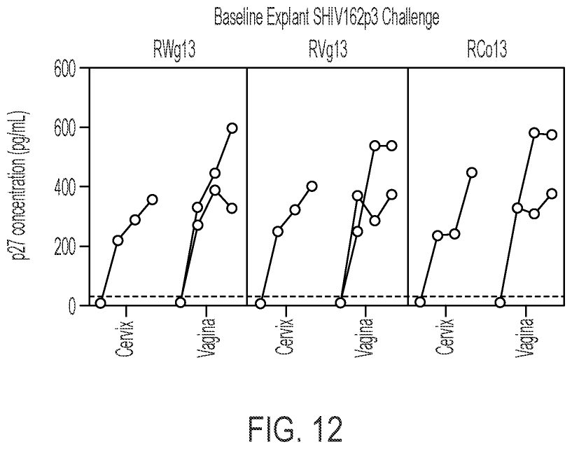

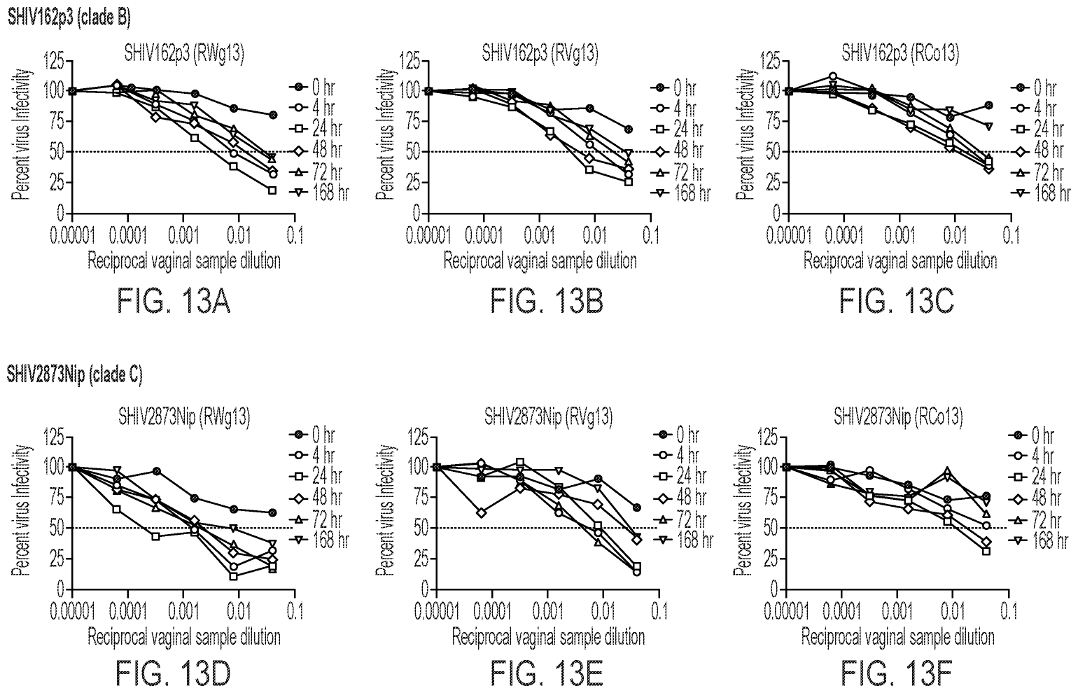

The next goal was to evaluate the pharmacokinetics of the anchored (aPGT121) and secreted (sPGT121) forms of PGT121 over one month in two physiologically relevant compartments—genital secretions and the FRT mucosa. It was hypothesized that aPGT121 would be retained in the secretions and mucosa longer than sPGT121. After delivery of two 750 pg doses of either aPGT121 or sPGT121 to sheep, genital secretions were collected longitudinally on a weekly basis and PGT121 concentrations were quantified via western blot (FIGS. 4A-4C and FIG. 11). The GPI anchor retained aPGT121 at high concentrations in genital secretions compared to sPGT121. Secretion kinetic patterns of aPGT121 were consistent: maximum bNAb concentration at 24 hours, followed by a plateau from 7 to 21 days, after which concentrations began to decrease (FIGS. 4A-B). At 28 days, the average aPGT121 concentration was 40 pg/mL. In contrast, sPGT121 concentrations peaked at 24 hours, then decreased to barely above 10 pg/mL at 14 days. The maximum mean concentration of PGT121 was achieved at 24 hours, with 210 pg/mL and 80 pg/mL for anchored and secreted PGT121, respectively.

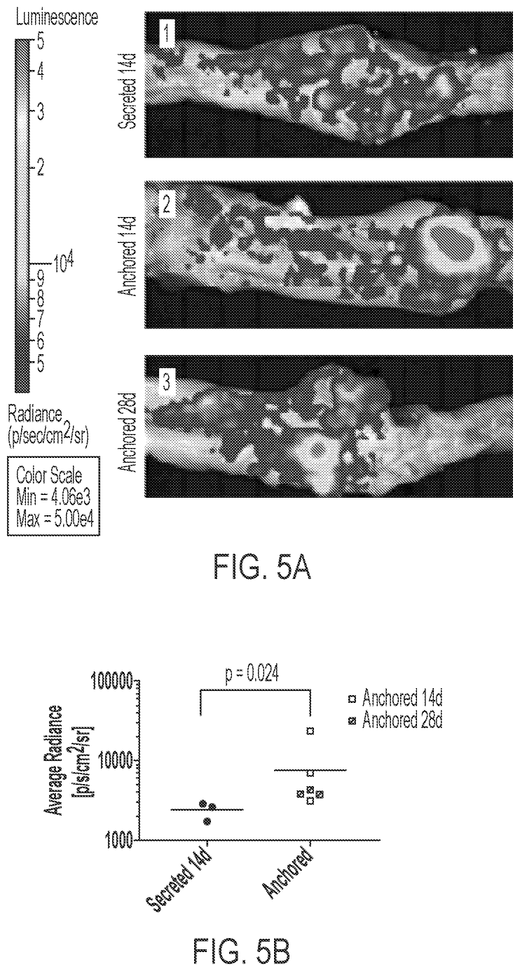

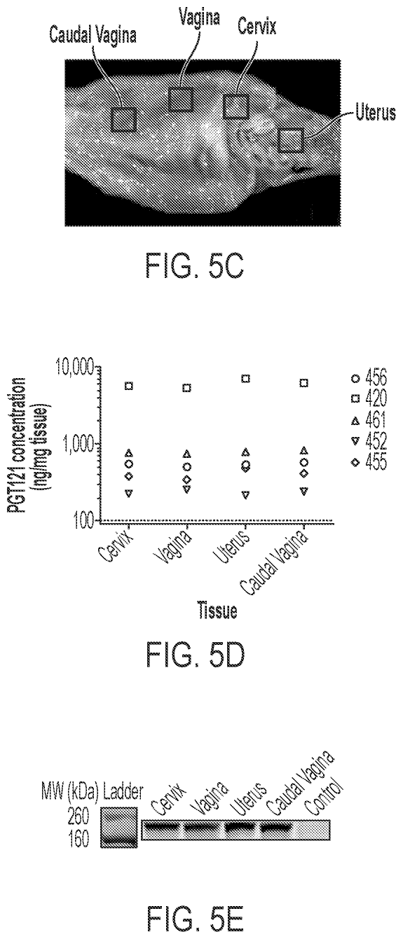

The distribution of PGT121 on the mucosal surface over time was evaluated. Anchoring PGT121 to the cell membrane surface retained the expressed antibody in the epithelium for at least 1-month post-transfection, while sPGT121 amounts were significantly reduced by two weeks (FIGS. 5A-5E). PGT121-NanoLuc® in the mucosal compartment was evaluated at 14 and 28 days post-transfection in two ways: (1) IVIS imaging of NanoLuc® signal directly following necropsy (FIGS. 5A-5B) and (2) quantitative western blot³⁶ following tissue pulverization (FIGS. 5C-5E). The luminescence signal in all aPGT121 tissues (n=6) was higher than in sPGT121 tissues (p=0.024). sPGT121 transfected animals at 28 days were originally scheduled but were repurposed, after assessing the low signal results at 14 days.

From each FRT explant taken at 28 days, regions of caudal vagina, rostral vagina, cervix, and uterus were removed, using the IVIS signal as a guide (FIGS. 5C). It was estimated by western blot that the average concentration of aPGT121 across the three animals was above 1 pg per mg of tissue (FIG. 5D). Surprisingly, similar quantities of antibody were measured in each section of the FRT, for a given animal (FIGS. 5D-E). Interestingly, while only rostral vagina and cervix were received doses of mRNA, aPGT121-NanoLuc® localized to other regions of the FRT, likely a result of DAF GPI linker re-association with cellular membranes after release.

### Example VI: PET/CT Imaging Detects mRNA in the FRT

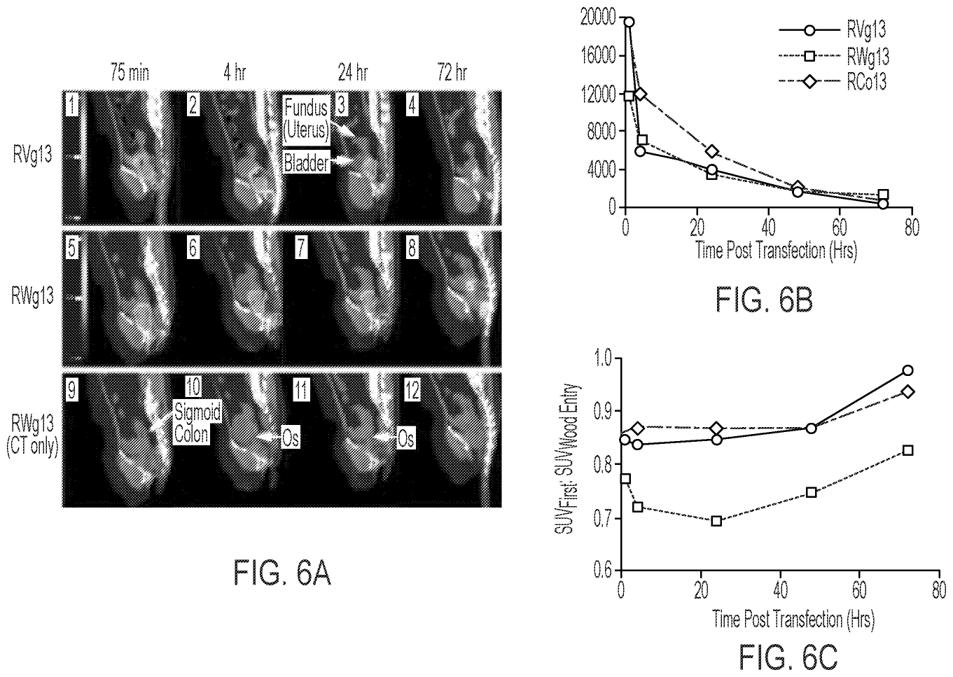

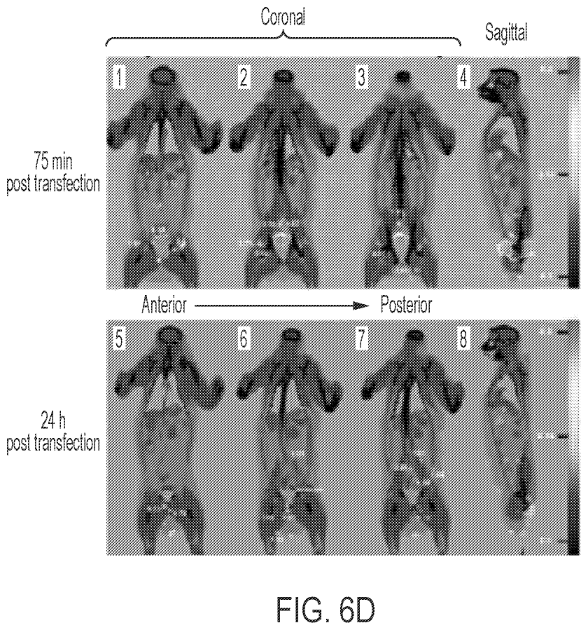

To characterize aerosolized mucosal mRNA trafficking and localization, PGT121 encoding mRNA was labeled orthogonally with ⁶⁴Cu, an approach described recently by our group. 250 pg of radiolabeled mRNA was delivered in two 125 pg doses, one at the ectocervix and a second dose after a 2-3 cm retraction of the atomizer within the vagina. Three rhesus macaques were treated in total. PET/CT imaging was performed at 70 minutes, 4 hours, 24 hours, 48 hours, and 72 hours post-administration (FIG. 6A-6D).

The visualization target during speculum insertion and mRNA delivery was the cervical os, which was the focal point of mRNA transfection, as resolved in the PET/CT imaging (FIG. 6A). Between 70 minutes and 4 hours, the PET signal in the FRT decreased by approximately 50-67% (FIG. 6B). After this initial rapid decrease, the rate of standard uptake value (SUV) decline from 4 hours to 72 hours decreased and resembled a more linear process (FIG. 6B). A potential cause of the rapid decrease over the first 4 hours is that excess mRNA in the delivery volume was excreted from the vagina. Excess mRNA is not surprising considering that the original aerosol parameters were optimized in sheep, and the vaginal vault of a rhesus macaque is smaller; therefore, aerosol parameters were adjusted for the remainder of the non-human primate studies. Hence, the radioactive signal also provided useful insight into the efficiency of the delivery method.

When the amount of radioactivity in the FRT (SUVFRT) was reported relative to the total radioactivity in the body (SUVWhole Body), then the relative amount of mRNA within the FRT remained constant over 48 hours and increased between 48 to 72 hours (FIG. 6C). This relative increase in mRNA concentration in the FRT is likely due to the SUV values in other organs, mainly draining lymph nodes (LNs) (FIG. 6D), decreasing below the limit of detection due to either mRNA or radioactive decay.

A large portion of the mRNA (>99%) was restricted to the FRT, while a small fraction of the mRNA was also detected via PET/CT in LNs 70 minutes after aerosolized delivery (FIG. 6D), including sacral, popliteal, inguinal, femoral, iliac, para-aortic, and mesenteric LNs. After 48 hours, the signal in the LNs was no longer detectable. However, signal was still abundant in the FRT mucosal surface after 72 hours (FIGS. 6A and 6C), indicating retention of mRNA within transfected cells.

### Example VII: Cervicovaginal Biopsy Explants are Protected From SHIV Challenge

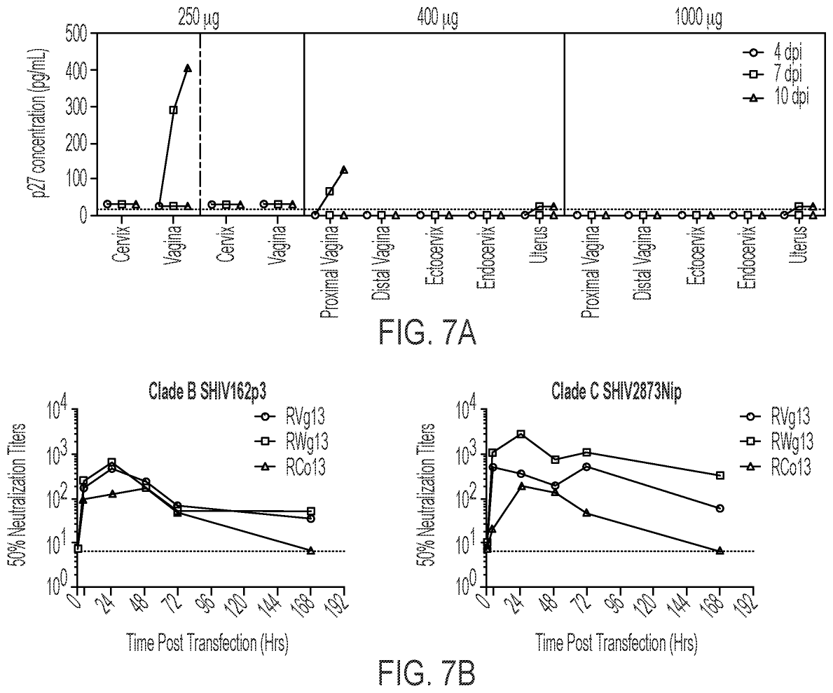

To test protective efficacy across the FRT, an ex vivo biopsy challenge model was used. Four female rhesus macaques were used: two were sprayed with 250 pg, one with 400 pg and one with 1000 μg of aPGT121 mRNA. To minimize potential leakage of excess mRNA solution, the dose volume was reduced to 150 μL. mRNA was delivered in two sequential doses, once at the ectocervix, then another dose 2-3 cm rostral in the vagina. Biopsies were collected before and at one day post-transfection. In the 250 μg group, one sample was taken from the endocervix and two from the vagina, while in the 400 and 1000 μg groups, two samples each were taken from the uterus, endocervix, ectocervix, proximal vagina, and distal vagina. FRT biopsies were then challenged with SHIV162p3, a Clade B Env virus. It was observed a dose-dependent decrease in p27 production in the biopsy explants (FIG. 7A). While one explant produced strong p27 at the 250 pg dose, this effect was diminished at 400 pg dose, and completely abrogated at the 1000 pg dose, where none of the ten biopsies from 5 different regions of the FRT yielded productive SHIV infection.

### Example VIII: Genital Secretions From aPGT121 mRNA Treated Macaques Neutralize SHIV In Vitro

To examine the functional activity of aPGT121, secretions from three biopsied macaques were collected prior to transfection and post-transfection with a 250 μg dose of aPGT121 mRNA at 4 hours, 24 hours, 48 hours, 72 hours, and 7 days. It is important to note that macaque RCo13 was transfected with an equal mass of only HC of aPGT121 mRNA. The secretions were evaluated for their ability to neutralize SHIV through the TZM-bl neutralization assay (FIGS. 7B and FIG. 12). Genital secretions from animals transfected with whole aPGT121 mRNA demonstrated an ability to neutralize SHIV162p3 (Clade B) and SHIV2873Nip (Clade C)³⁹ out to 1 week. Critically, by 4 hours post-transfection, genital secretions already displayed substantial neutralizing activity. The kinetics over time for the heavy chain only (macaque RCo13) construct suggest that without the association of the constant regions of the HC and LC, the binding pocket is compromised, resulting in reduced neutralization.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

We claim:

1-30. (canceled)

31. A PGT121 antibody or an antigen binding fragment thereof comprising a GPI membrane anchor, wherein the antibody or antigen binding fragment thereof specifically binds to a gp120 protein and thereby reduces the infectivity of the HIV virus or HIV virus particle.

32. The antibody or an antigen binding fragment according to claim 31 comprising a heavy chain encoded by SEQ ID NO:1 and a light chain encoded by SEQ ID NO:2.

33. The antibody or an antigen binding fragment of claim 31, wherein the GPI anchor is in a heavy chain of the antibody.

34. The antibody or antigen-binding fragment of claim 31 encoded by a recombinant genetic construct.

35. The recombinant genetic construct of claim 34, wherein the construct is a mRNA genetic construct.

36. The recombinant genetic construct of claim 34, wherein the construct is fully modified with complete substitution of uridine with n1-methyl-pseudouridine.

37. A method for prophylactically inhibiting or reducing HIV infection in a female subject in need thereof comprising administering to the female subject a pharmaceutical composition comprising an mRNA construct encoding for a PGT121 antibody or antigen binding fragment thereof and at least one pharmaceutically acceptable excipient; and
wherein the construct specifically binds to a gp120 protein and thereby reduces the infectivity of the HIV virus or HIV virus particle.

38. The method of claim 37, wherein the pharmaceutical composition is administered by transfecting the cervicovaginal epithelial cells in the subject with the mRNA construct via an aerosol in an amount effective to reduce or inhibit HIV infection in the subject.

39. The method of claim 38, wherein the mRNA construct is a vector encoding an antibody or an antigen binding fragment thereof comprising a heavy chain nucleic acid sequence with 90%, 95%, 99% or 100% sequence identity to any one of SEQ ID NOs: 9 and 10, a GPI membrane anchor have the nucleic acid sequence with 90%, 95%, 99% or 100% sequence identity to any one of SEQ ID NOs: 11, a light chain nucleic acid sequence with 90%, 95%, 99% or 100% sequence identity to any one of SEQ ID NOs: 12 and 13.

40. The method of claim 38, wherein the mRNA construct is a vector encoding an antibody or an antigen binding fragment thereof comprising a heavy chain nucleic acid sequence with 90%, 95%, 99% or 100% sequence identity to any one of SEQ ID NOs: 14, 15, 16, and 17, a GPI membrane anchor have the nucleic acid sequence with 90%, 95%, 99% or 100% sequence identity to any one of SEQ ID NOs: 18, a light nucleic acid sequence with 90%, 95%, 99% or 100% sequence identity to any one of SEQ ID NOs: 19, 20, 21, and 22.

41. The method of claim 38, wherein the mRNA construct is a vector encoding an antibody or an antigen binding fragment thereof comprising a heavy chain nucleic acid sequence with 90%, 95%, 99% or 100% sequence identity to any one of SEQ ID NOs: 14, 15, 16, and 17, a GPI membrane anchor have the nucleic acid sequence with 90%, 95%, 99% or 100% sequence identity to any one of SEQ ID NOs: 18, a light nucleic acid sequence with 90%, 95%, 99% or 100% sequence identity to any one of SEQ ID NOs: 19, 20, 21, and 22.

42. The method of claim 38, wherein administration via aerosol is delivered by an aerosol delivery device.

43. The aerosol delivery device of claim 42, wherein the delivery device is an atomizer or a dual-chamber syringe containing lyophilized mRNA and water and an atomizer suitable for self-insertion into the FRT.

44. A monoclonal antibody or an antigen binding fragment thereof comprising at least one heavy chain amino acid sequence, a GPI membrane anchor having an amino acid sequence, and at least one light chain amino acid.

45. The monoclonal antibody or an antigen binding fragment of claim 44, wherein the mRNA construct is a vector comprising a heavy chain nucleic acid sequence with 90%, 95%, 99% or 100% sequence identity to any one of SEQ ID NOs: 23 and 24, a GPI membrane anchor have the nucleic acid sequence with 90%, 95%, 99% or 100% sequence identity to any one of SEQ ID NOs: 25.

46. The monoclonal antibody or an antigen binding fragment of claim 44 comprising a heavy chain amino acid sequence according to SEQ ID NO:27, a GPI membrane anchor have the amino acid sequence according to SEQ ID NO:28, and a light chain amino acid sequence according to SEQ ID NO:30.

47. The monoclonal antibody or antigen binding fragment of claim 44 having a first heavy chain with an amino acid sequence according to SEQ ID NO:32, a second heavy chain having an amino acid sequence according to SEQ ID NO:34, a GPI membrane anchor with an amino acid sequence according to 35, a first light chain with an amino acid sequence according to 37, and a second light chain having an amino acid sequence according to SEQ ID NO:39.

48. The monoclonal antibody or antigen binding fragment of claim 44 having a heavy chain with an amino acid sequence according to SEQ ID NO:41 and a GPI membrane anchor according to SEQ ID NO:42.


SEQUENCE LISTING (52 sequences)

SEQ ID NO: 1 - DNA, 1437 bp, Artificial Sequence
  source: /note=“Description of Artificial Sequence Synthetic polynucleotide”
cgattggcggaaggccgtcaaggccgcatttttaagctttaatacgactcactatagggaaataagagagaaaagaagagtaagaagaaatataagagccaccatgaagtgggtgaccttcatcagcctgctgttcctgttcagcagcgcctacagcagcgacatcagcgtggcccccggcgagaccgccagaatcagctgcggcgagaagagcctgggcagcagagccgtgcagtggtaccagcacagagccggccaggcccccagcctgatcatctacaacaaccaggacagacccagcggcatccccgagagattcagcggcagccccgacagccccttcggcaccaccgccaccctgaccatcaccagcgtggaggccggcgacgaggccgactactactgccacatctgggacagcagagtgcccaccaagtgggtgttcggcggcggcaccaccctcaccgtgctgagagccgtggccgcccccagcgtgttcatcttcccccccagcgaggaccaggtgaagagcggcaccgtgagcgtggtgtgcctgctgaacaacttctaccccagagaggccagcgtgaagtggaaggtggacggcgtgctgaagaccggcaacagccaggagagcgtgaccgagcaggacagcaaggacaacacctacagcctgagcagcaccctgaccctgagcagcaccgactaccagagccacaacgtgtacgcctgcgaggtgacccaccagggcctgagcagccccgtgaccaagagcttcaacagaggcgagtgcggaggcgggggcagcgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtgataagctgccttctgcggggcttgccttctggccatgcccttcttctctcccttgcacctgtacctcttggtctttgaataaagcctgagtaggaaggcggccgcaaaaactgggcc

SEQ ID NO: 2 - DNA, 1758 bp, Artificial Sequence
  source: /note=“Description of Artificial Sequence Synthetic polynucleotide”
ttttaagctttaatacgactcactatagggaaataagagagaaaagaagagtaagaagaaatataagagccaccatgaaatgggtcacctttatcagcctgctgttcctgttcagcagcgcctacagccagatgcagctccaagagtctggccctggcctggtcaagcctagcgaaacactgagcctgacctgttccgtgtccggcgccagcatcagcgatagctactggtcctggatcagaagaagccccggcaaaggcctggaatggatcggctacgtgcacaagagcggcgacaccaactatagccccagcctgaagtccagagtgaacctgagcctggacaccagcaagaaccaggtgtccctgtctctggtggccgccacagctgctgatagcggcaagtactactgcgccagaacactgcacggcagacggatctatggcatcgtggccttcaacgagtggttcacctacttctacatggacgtgtggggcaacggcacccaagtgacagtgtctagcgccagcacaaagggccctagcgttttcccactggctcccagcagcagaagcaccagcgaatctacagccgctctgggctgcctcgtgaaggactactttcctgagccagtgaccgtgtcctggaacagcggctctctgacatctggcgtgcacacctttccagccgtgctgcaaagcagcggcctgtactctctgagcagcgtggtcacagtgcctagctctagcctgggcacccagacctacgtgtgcaatgtgaatcacaagcccagcaacaccaaggtggacaagagagtggaaatcaagacctgcggcggaggcagcaagcctcctacatgtcctccatgtcctgctccagaagctgccggcggaccttccgtgtttctgttccctccaaagcctaaggacaccctgatgatcagcagaacccctgaagtgacctgcgtggtggtggacgtgtcccaagaggaccccgacgtgaagttcaattggtacgtgaacggcgccgaggtgcaccacgctcagacaaagccaagagagacacagtacaacagcacctacagagtggtgtccgtgctgaccgtgacacaccaggattggctgaacggcaaagagtacacctgtaaagtctccaacaaggccctgcctgctcctatccagaaaaccatcagcaaggacaagggccagcctcgcgaaccccaggtttacacactgcctccaagcagagaggaactgaccaaaaatcaggtttccctgacctgcctggttaagggcttctaccccagcgacatcgtggtggaatgggagtctagcggacagcccgagaacacctacaagaccacacctccagtgctggacagcgacggcagctacttcctgtacagcaagctgacagtggacaagtccagatggcagcagggcaacgtgttcagctgcagcgtgatgcacgaggccctgcacaaccactacacccagaagtctctgagcgtcagccctggcaagcacgagacaacccctaacaaaggcagcggcaccacctctggcaccacaagactgctgtctggccacacctgtttcacactgaccggcctgctgggcacactggttacaatgggactgctgacctgataagctgccttctgcggggcttgccttctggccatgcccttcttctctcccttgcacctgtacctcttggtctttgaataaagcctgagtaggaaggcggccgcaaaaa

SEQ ID NO: 3 - DNA, 40 bp, Artificial Sequence
  source: /note=“Description of Artificial Sequence Synthetic primer”
gcgacgattggcggaaggccgtcaaggccgcatttttaag

SEQ ID NO: 4 - DNA, 38 bp, Artificial Sequence
  source: /note=“Description of Artificial Sequence Synthetic primer”
cgggcagtgagcggaaggcccatgaggcccagtttttg

SEQ ID NO: 5 - DNA, 18 bp, Artificial Sequence
  source: /note=“Description of Artificial Sequence Synthetic primer”
ggccttccgctcactgcc

SEQ ID NO: 6 - DNA, 18 bp, Artificial Sequence
  source: /note=“Description of Artificial Sequence Synthetic primer”
ggccttccgccaatcgtc

SEQ ID NO: 7 - DNA, 25 bp, Artificial Sequence
  source: /note=“Description of Artificial Sequence Synthetic primer”
taaaacgacggccagtgagcgcgac

SEQ ID NO: 8 - DNA, 25 bp, Artificial Sequence
  source: /note=“Description of Artificial Sequence Synthetic primer”
ctggcacgacaggtttcccgactgg

SEQ ID NO: 9 - DNA, 51 bp, Artificial Sequence
  source: /note=“Description of Artificial Sequence Synthetic oligonucleotide”
atgaaatgggtcacctttatcagcctgctgttcctgttcagcagcgcctac

SEQ ID NO: 10 - DNA, 1398 bp, Artificial Sequence
  source: /note=“Description of Artificial Sequence Synthetic polynucleotide”
agccagatgcagctccaagagtctggccctggcctggtcaagcctagcgaaacactgagcctgacctgttccgtgtccggcgccagcatcagcgatagctactggtcctggatcagaagaagccccggcaaaggcctggaatggatcggctacgtgcacaagagcggcgacaccaactatagccccagcctgaagtccagagtgaacctgagcctggacaccagcaagaaccaggtgtccctgtctctggtggccgccacagctgctgatagcggcaagtactactgcgccagaacactgcacggcagacggatctatggcatcgtggccttcaacgagtggttcacctacttctacatggacgtgtggggcaacggcacccaagtgacagtgtctagcgccagcacaaagggccctagcgttttcccactggctcccagcagcagaagcaccagcgaatctacagccgctctgggctgcctcgtgaaggactactttcctgagccagtgaccgtgtcctggaacagcggctctctgacatctggcgtgcacacctttccagccgtgctgcaaagcagcggcctgtactctctgagcagcgtggtcacagtgcctagctctagcctgggcacccagacctacgtgtgcaatgtgaatcacaagcccagcaacaccaaggtggacaagagagtggaaatcaagacctgcggcggaggcagcaagcctcctacatgtcctccatgtcctgctccagaagctgccggcggaccttccgtgtttctgttccctccaaagcctaaggacaccctgatgatcagcagaacccctgaagtgacctgcgtggtggtggacgtgtcccaagaggaccccgacgtgaagttcaattggtacgtgaacggcgccgaggtgcaccacgctcagacaaagccaagagagacacagtacaacagcacctacagagtggtgtccgtgctgaccgtgacacaccaggattggctgaacggcaaagagtacacctgtaaagtctccaacaaggccctgcctgctcctatccagaaaaccatcagcaaggacaagggccagcctcgcgaaccccaggtttacacactgcctccaagcagagaggaactgaccaaaaatcaggtttccctgacctgcctggttaagggcttctaccccagcgacatcgtggtggaatgggagtctagcggacagcccgagaacacctacaagaccacacctccagtgctggacagcgacggcagctacttcctgtacagcaagctgacagtggacaagtccagatggcagcagggcaacgtgttcagctgcagcgtgatgcacgaggccctgcacaaccactacacccagaagtctctgagcgtcagccctggcaag

SEQ ID NO: 11 - DNA, 123 bp, Artificial Sequence
  source: /note=“Description of Artificial Sequence Synthetic polynucleotide”
cacgagacaacccctaacaaaggcagcggcaccacctctggcaccacaagactgctgtctggccacacctgtttcacactgaccggcctgctgggcacactggttacaatgggactgctgacc

SEQ ID NO: 12 - DNA, 51 bp, Artificial Sequence
  source: /note=“Description of Artificial Sequence Synthetic oligonucleotide”
atgaagtgggtgaccttcatcagcctgctgttcctgttcagcagcgcctac

SEQ ID NO: 13 - DNA, 639 bp, Artificial Sequence
  source: /note=“Description of Artificial Sequence Synthetic polynucleotide”
agcagcgacatcagcgtggcccccggcgagaccgccagaatcagctgcggcgagaagagcctgggcagcagagccgtgcagtggtaccagcacagagccggccaggcccccagcctgatcatctacaacaaccaggacagacccagcggcatccccgagagattcagcggcagccccgacagccccttcggcaccaccgccaccctgaccatcaccagcgtggaggccggcgacgaggccgactactactgccacatctgggacagcagagtgcccaccaagtgggtgttcggcggcggcaccaccctcaccgtgctgagagccgtggccgcccccagcgtgttcatcttcccccccagcgaggaccaggtgaagagcggcaccgtgagcgtggtgtgcctgctgaacaacttctaccccagagaggccagcgtgaagtggaaggtggacggcgtgctgaagaccggcaacagccaggagagcgtgaccgagcaggacagcaaggacaacacctacagcctgagcagcaccctgaccctgagcagcaccgactaccagagccacaacgtgtacgcctgcgaggtgacccaccagggcctgagcagccccgtgaccaagagcttcaacagaggcgagtgc

SEQ ID NO: 14 - DNA, 57 bp, Artificial Sequence
  source: /note=“Description of Artificial Sequence Synthetic oligonucleotide”
atgggatggtcctgcatcatcctgttcctggtggccaccgccaccggcgtgcacagc

SEQ ID NO: 15 - DNA, 1383 bp, Artificial Sequence
  source: /note=“Description of Artificial Sequence Synthetic polynucleotide”
gaggtgcgcctcgtggagagcggcggaggcctggtgaagccgggcggcagcctgcggctgagctgtagcgccagcggctttaacttcgacgacgcctggatgacatgggtgcggcagcctcctggcaagggcctggaatgggtcggcagaatcagtggccctggcgagggctggtctgtggattacgctgagtctgtgaagggcagattcaccatcagcagaaagaatagcaagaacacactgtacctggaaatgaacaacgtgcggaccgaggataccggatattacttctgcgccagaaccggcaagcactacgacttctggagcggctaccctcctggggaggaatactttcaggactggggccaaggcacaaaggtgatcgtgagcagcgcctctaccaaaggcccctccgtgttcccactggctcctagctctaagagcaccagcggcggcacagccgctcttggatgtctggtcaaggactacttccctgagcctgttacagtgtcctggaacagcggggccctgaccagcggcgtgcacaccttccccgccgtactgcagagcagtggactgtatagcctctccagcgttgtgaccgtgcccagctccagcctgggcacccagacctacatctgcaatgtgaaccacaagccatctaacaccaaggtggacaagaaggtggaacccaaatcttgcgacaagacccacacatgtcctccttgccctgcccctgagttcgagggcggaccttctgtgtttctgtttccacctaagcctaaggacaccctgatgatctctagaacccctgaggtgacctgcgtggtggtggacgtgtctcacgaggaccccgaggtgaaattcaactggtacgtggatggcgtggaagtgcacaacgccaagacaaaacccagagaggagcagtacaacagcacataccgggtggtgtccgtcttgaccgtgctgcaccaggattggctgaacggcaaggaatacaagtgcaaggtcagcaataaggccctgcctgctagcatcgagaagaccatttctaaagccaagggccaacctcgggaaccccaggtgtacaccctgcctccatgtagagatgaactgacaaagaaccaggtgtccctgtggtgcctggtgaagggcttctacccctctgatatcgccgtggaatgggagagcaatggccagcccgaaaacaattacaaaaccacccctccagtgctggacagcgacggcagcttcttcctgtacagcaagctgaccgtggacaagagcagatggcagcagggcaacgtgttcagctgcagcgtgctgcatgaggccctgcacagccactatacccagaaatctctgagcctgagccccggaaaa

SEQ ID NO: 16 - DNA, 57 bp, Artificial Sequence; identical to SEQ ID NO: 14
  source: /note=“Description of Artificial Sequence Synthetic oligonucleotide”

SEQ ID NO: 17 - DNA, 1368 bp, Artificial Sequence
  source: /note=“Description of Artificial Sequence Synthetic polynucleotide”
caggtgcagctgcaacagagcggccccgaagtggtcaaaccaggcgccagcgtgaaaatgagctgtaaggctagtggatacacctttaccagctacgtgatccactgggtgcggcagaaacctggccagggcctggactggatcggctacatcaacccctacaacgacggcactgattacgacgagaagttcaagggcaaggccacactgacctccgacacaagcacatccaccgcttatatggaactgagcagcctgagaagcgaggacaccgcagtgtactactgcgccagagaaaaagataactacgccaccggagcctggttcgcctactggggccagggcacactggttaccgtctctagcgccagcaccgccgcccctagcgttttcatcttccctcctagcgacgagcaactgaagagcggcaccgctagcgtcgtgtgcctgctgaacaacttctaccctcgggaagccaaggtccagtggaaggtggacaacgccctgcagagcggaaatagccaggagtctgtgacggaacaagatagcaaggacagcacatactccctgagcagcacactgaccctgagcaaggccgattacgagaagcacaaggtgtatgcctgtgaagtgacccaccagggcctgtcttctcctgtgaccaagagcttcaatagaggcgagtgcgacaagacccacacctgtccaccgtgccccgctcccgagttcgagggcggccctagcgtgtttctgtttccacctaagcccaaggataccctgatgatctctagaacacctgaggtgacatgtgtggtggtggacgtgagccacgaggatcctgaggtgaaattcaactggtacgtggatggcgtggaagtgcacaacgccaaaacaaaacccagagaggagcagtacaattctacctaccgggtggtgtccgtgctgaccgtgctgcaccaggactggctgaacggcaaagagtacaagtgcaaggtgtccaacaaggccctgcctgcctctatcgagaagaccatcagcaaggctaagggacaacctagagaacctcaggtttgcaccctgcctccttcccgcgacgaactgaccaagaaccaggtgagtctgtcttgcgccgtgaagggcttctaccccagcgacatcgctgtggaatgggagagcaacggccagcccgagaacaactataagacaacccctcctgtgctggatagcgacggcagctttttcctggtgagcaagctgaccgtggacaagagcagatggcagcagggcaacgtgttcagctgcagcgtgctgcatgaggccctgcacagccactatacccagaaatctctgagcctgagccccggaaaa

SEQ ID NO: 18 - DNA, 123 bp, Artificial Sequence
  source: /note=“Description of Artificial Sequence Synthetic polynucleotide”
catgagacaacccctaacaagggctctggaacaacaagcggaacaaccagactgctgagcggccacacatgcttcaccctgaccggcctgctgggaacactggtgaccatgggcctgctgact

SEQ ID NO: 19 - DNA, 51 bp, Artificial Sequence
  source: /note=“Description of Artificial Sequence Synthetic oligonucleotide”
atgggctggtcctgcatcatcctgttcctggtggccaccgccacaggcagc

SEQ ID NO: 20 - DNA, 651 bp, Artificial Sequence
  source: /note=“Description of Artificial Sequence Synthetic polynucleotide”
tgggccgcttctgagctgacacaggaccccgccgtgtccgtggccctgaagcaaacagtgaccatcacttgtagaggcgacagcctgagaagccactacgccagctggtaccagaaaaagaccggccaggcccctaagctgctgttctacggcaagaacaatcgccccagcggagtgcccgacagattcagcggaagcgcctctggcaaccgggcctctctgaccatcagcggagcccaggctgaagatgacgccgagtactactgcagcagcagagataagtccggctctagactgagcgtgttcggcggcggaacaaagctgaccgtgctgagccaacctaaggctgcccctagcgtgaccctgtttccacctagctccgaggaactccaggccaacaaggccaccctggtctgcctgatcagcgacttctatcctggcgccgtgaccgtcgcctggaaagctgatagcagccctgtgaaggccggcgtggaaaccaccaccccttcaaagcagagcaacaacaaatacgccgcttcttcttacctgtctctgacccccgagcagtggaagtcccaccggagctacagctgccaggtgacacacgagggcagcaccgtggaaaagacagttgcaccaacagagtgtagc

SEQ ID NO: 21 - DNA, 51 bp, Artificial Sequence
  source: /note=“Description of Artificial Sequence Synthetic oligonucleotide”
atgggctggtcgtgcatcatcctgtttctggtggccacagctacaggctct

SEQ ID NO: 22 - DNA, 657 bp, Artificial Sequence
  source: /note=“Description of Artificial Sequence Synthetic polynucleotide”
tgggccgacattgtgatgacccaaagccctgacagcctggctgtgtctctcggcgaacgggtgacaatgaactgcaagagcagccagtccctgctgtatagcaccaaccagaaaaactacctggcctggtatcagcagaagcccggccagtctcctaagctgctgatctactgggcttctaccagagagagcggtgttcctgatagattcagcggcagcggcagcggcactgatttcaccctgaccatcagcagcgtgcaggccgaggacgtggccgtgtactactgccagcaatactacagctacagaaccttcggcggaggcacaaaactggaaatcaagagctccgccagcaccaagggcccttctgtgttcccactggccccaagctctaagtccacctccggaggaaccgccgccctgggctgtctggtcaaggactacttccctgagcctgtgacagtgtcctggaatagcggcgccctgaccagcggagtgcacacctttcccgccgtgctgcagagctcaggcctgtacagcctttctagcgtggtgacagtgcccagcagcagcctgggcacacagacctacatctgcaacgtgaaccacaagcctagcaataccaaagtggacaagaaggtcgagcccaagtcttgt

SEQ ID NO: 23 - DNA, 54 bp, Artificial Sequence
  source: /note=“Description of Artificial Sequence Synthetic oligonucleotide”
atgaaatgggtcacattcatctctctgctgttcctgttcagcagcgcctatagc

SEQ ID NO: 24 - DNA, 363 bp, Artificial Sequence
  source: /note=“Description of Artificial Sequence Synthetic polynucleotide”
gaggtgcagctggtggaaagcggcggaggcctcgtgcaagctggaggatttctgagactgagctgcgagctgcggggcagcatcttcaaccagtacgccatggcctggttccggcaggcccctggcaaggaacgggaattcgtggccggcatgggagctgtgccccactacggcgagttcgttaaaggcagattcaccatctctagagataatgccaagtccaccgtgtacctgcagatgagcagcctgaagcctgaggacaccgccatctacttctgcgccagaagcaagagcacatacatcagctacaacagcaacggctacgactactggggccgcggcacacaggtgaccgtgtccagc

SEQ ID NO: 25 - DNA, 138 bp, Artificial Sequence
  source: /note=“Description of Artificial Sequence Synthetic polynucleotide”
ggcggcggcggctctcacgagacaaccccaaacaagggttccggcaccaccagcggaaccaccagactgctgtctggccacacctgttttaccctgacaggcctgctgggcaccctggtgacaatgggcctgctgaca

SEQ ID NO: 26 - protein, 17 aa, Artificial Sequence
  source: /note=“Description of Artificial Sequence Synthetic peptide”
MKWVTFISLLFLFSSAY

SEQ ID NO: 27 - protein, 466 aa, Artificial Sequence
  source: /note=“Description of Artificial Sequence Synthetic polypeptide”
SQMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSSASTKGPSVFPLAPSSRSTSESTAALGCLVKDYFPEPVTVSWNSGSLTSGVHTFPAVLQSSGLYSLSSVVTVPSSSLGTQTYVCNVNHKPSNTKVDKRVEIKTCGGGSKPPTCPPCPAPEAAGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVMHEALHNHYTQKSLSVSPGK

SEQ ID NO: 28 - protein, 41 aa, Artificial Sequence
  source: /note=“Description of Artificial Sequence Synthetic polypeptide”
HETTPNKGSGTTSGTTRLLSGHTCFTLTGLLGTLVTMGLLT

SEQ ID NO: 29 - protein, 17 aa, Artificial Sequence
  source: /note=“Description of Artificial Sequence Synthetic peptide”
MKWVTFISLLFLFSSAY

SEQ ID NO: 30 - protein, 213 aa, Artificial Sequence
  source: /note=“Description of Artificial Sequence Synthetic polypeptide”
SSDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVLRAVAAPSVFIFPPSEDQVKSGTVSVVCLLNNFYPREASVKWKVDGVLKTGNSQESVTEQDSKDNTYSLSSTLTLSSTDYQSHNVYACEVTHQGLSSPVTKSFNRGEC

SEQ ID NO: 31 - protein, 19 aa, Artificial Sequence
  source: /note=“Description of Artificial Sequence Synthetic peptide”
MGWSCIILFLVATATGVHS

SEQ ID NO: 32 - protein, 461 aa, Artificial Sequence
  source: /note=“Description of Artificial Sequence Synthetic polypeptide”
EVRLVESGGGLVKPGGSLRLSCSASGFNFDDAWMTWVRQPPGKGLEWVGRISGPGEGWSVDYAESVKGRFTISRKNSKNTLYLEMNNVRTEDTGYYFCARTGKHYDFWSGYPPGEEYFQDWGQGTKVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 33 - protein, 19 aa, Artificial Sequence
  source: /note=“Description of Artificial Sequence Synthetic peptide”
MGWSCIILFLVATATGVHS

SEQ ID NO: 34 - protein, 456 aa, Artificial Sequence
  source: /note=“Description of Artificial Sequence Synthetic polypeptide”
QVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSSASTAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHSHYTQKSLSLSPGK

SEQ ID NO: 35 - protein, 41 aa, Artificial Sequence; identical to SEQ ID NO: 28
  source: /note=“Description of Artificial Sequence Synthetic polypeptide”

SEQ ID NO: 36 - protein, 17 aa, Artificial Sequence
  source: /note=“Description of Artificial Sequence Synthetic peptide”
MGWSCIILFLVATATGS

SEQ ID NO: 37 - protein, 217 aa, Artificial Sequence
  source: /note=“Description of Artificial Sequence Synthetic polypeptide”
WAASELTQDPAVSVALKQTVTITCRGDSLRSHYASWYQKKTGQAPKLLFYGKNNRPSGVPDRFSGSASGNRASLTISGAQAEDDAEYYCSSRDKSGSRLSVFGGGTKLTVLSQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 38 - protein, 17 aa, Artificial Sequence
  source: /note=“Description of Artificial Sequence Synthetic peptide”
MGWSCIILFLVATATGS

SEQ ID NO: 39 - protein, 219 aa, Artificial Sequence
  source: /note=“Description of Artificial Sequence Synthetic polypeptide”
WADIVMTQSPDSLAVSLGERVTMNCKSSQSLLYSTNQKNYLAWYQQKPGQSPKLLIYWASTRESGVPDRFSGSGSGTDFTLTISSVQAEDVAVYYCQQYYSYRTFGGGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 40 - protein, 18 aa, Artificial Sequence
  source: /note=“Description of Artificial Sequence Synthetic peptide”
MKWVTFISLLFLFSSAYS

SEQ ID NO: 41 - protein, 121 aa, Artificial Sequence
  source: /note=“Description of Artificial Sequence Synthetic polypeptide”
EVQLVESGGGLVQAGGFLRLSCELRGSIFNQYAMAWFRQAPGKEREFVAGMGAVPHYGEFVKGRFTISRDNAKSTVYLQMSSLKPEDTAIYFCARSKSTYISYNSNGYDYWGRGTQVTVSS

SEQ ID NO: 42 - protein, 46 aa, Artificial Sequence
  source: /note=“Description of Artificial Sequence Synthetic polypeptide”
GGGGSHETTPNKGSGTTSGTTRLLSGHTCFTLTGLLGTLVTMGLLT

SEQ ID NO: 43 - DNA, 24 bp, Artificial Sequence
  source: /note=“Description of Artificial Sequence Synthetic oligonucleotide”
  source: /note=“Description of Combined DNA/RNA Molecule Synthetic oligonucleotide”
tttttttgcaagccccgcagaagt

SEQ ID NO: 44 - DNA, 25 bp, Artificial Sequence
  source: /note=“Description of Artificial Sequence Synthetic oligonucleotide”
  source: /note=“Description of Combined DNA/RNA Molecule Synthetic oligonucleotide”
ttttatttagagaagaagggcatgg

SEQ ID NO: 45 - protein, 105 aa, Artificial Sequence
  source: /note=“Description of Artificial Sequence Synthetic polypeptide”
  VARIANT (2)..(2): /replace=“Phe” or “Ser”
  VARIANT (3)..(3): /replace=“Val” or “Met”
  VARIANT (61)..(61): /replace=“Thr” or “Arg”
  VARIANT (90)..(90): /replace=“Arg” or “Gly”
  VARIANT (98)..(98): /replace=“Glu” or “Arg”
  SITE (1)..(105): /note=“Variant residues given in the sequence have no preference with respect to those in the annotations for variant positions”
SDISVAPGETARISCGEESLGSRAVQWYQQRPGQAPSLIIYNNQDRPSGIPERFSGSPDSPFGTTATLTITSVEAGDEADYYCHIWDSRVPTNWVFGGGTTLTVL

SEQ ID NO: 46 - protein, 105 aa, Artificial Sequence
  source: /note=“Description of Artificial Sequence Synthetic polypeptide”
SDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVL

SEQ ID NO: 47 - protein, 105 aa, Artificial Sequence
  source: /note=“Description of Artificial Sequence Synthetic polypeptide”
TFVSVAPGQTARITCGEESLGSRSVIWYQQRPGQAPSLIIYNNNDRPSGIPDRFSGSPGSTFGTTATLTITSVEAGDEADYYCHIWDSRRPTNWVFGEGTTLIVL

SEQ ID NO: 48 - protein, 105 aa, Artificial Sequence
  source: /note=“Description of Artificial Sequence Synthetic polypeptide”
SSMSVSPGETAKISCGKESIGSRAVQWYQQKPGQPPSLIIYNNQDRPAGVPERFSASPDFRPGTTATLTITNVDAEDEADYYCHIYDARGGTNWVFDRGTTLTVL

SEQ ID NO: 49 - protein, 132 aa, Artificial Sequence
  source: /note=“Description of Artificial Sequence Synthetic polypeptide”
  VARIANT (2)..(2): /replace=“Val” or “Leu”
  VARIANT (30)..(30): /replace=“Arg” or “Asn”
  VARIANT (32)..(32): /replace=“Asn” or “Ala”
  VARIANT (44)..(44): /replace=“Gln” or “Arg”
  VARIANT (53)..(53): /replace=“Asp” or “His”
  VARIANT (68)..(68): /replace=“His” or “Thr”
  VARIANT (77)..(77): /replace=“Leu” or “Glu”
  VARIANT (81)..(81): /replace=“Arg” or “Lys”
  VARIANT (84)..(84): /replace=“Gly” or “Asp”
  VARIANT (85)..(85): /replace=“Val” or “Leu”
  VARIANT (92)..(92): /replace=“Ile” or “Thr”
  VARIANT (111)..(111): /replace=“Lys” or “Gly”
  VARIANT (127)..(127): /replace=“Ser” or “Ala”
  SITE (1)..(132): /note=“Variant residues given in the sequence have no preference with respect to those in the annotations for variant positions”
QMHLQESGPGLVKPSETLSLTCSVSGASISDSYWSWIRQSPGKGPEWIGYVHKSGDTNYNPSLKSRVNLSLDTSKNQVSLSLVAATAADSAKYYCARTLHGRRIYGIVAFNEWFTYFYMDVWGKGTQVTVSS

SEQ ID NO: 50 - protein, 132 aa, Artificial Sequence
  source: /note=“Description of Artificial Sequence Synthetic polypeptide”
QMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSS

SEQ ID NO: 51 - protein, 132 aa, Artificial Sequence
  source: /note=“Description of Artificial Sequence Synthetic polypeptide”
QVHLQESGPGLVKPSETLSLTCNVSGTLVRDNYWSWIRQPLGKQPEWIGYVHDSGDTNYNPSLKSRVHLSLDKSKNLVSLRLTGVTAADSAIYYCATTKHGRRIYGVVAFKEWFTYFYMDVWGKGTSVTVSS

SEQ ID NO: 52 - protein, 132 aa, Artificial Sequence
  source: /note=“Description of Artificial Sequence Synthetic polypeptide”
QLHLQESGPGLVKPPETLSLTCSVSGASINDAYWSWIRQSPGKRPEWVGYVHHSGDTNYNPSLKRRVTFSLDTAKNEVSLKLVDLTAADSATYFCARALHGKRIYGIVALGELFTYFYMDVWGKGTAVTVSS